(19) United States
(12) Patent Application Publication
(10) Pub. No.: US 2025/0367085 A1
(43) Pub. Date: Dec. 4, 2025

(54) COSMETIC COMPOSITION FOR IMMEDIATELY IMPROVING THE APPEARANCE OF SKIN

(71) Applicant: L'OREAL, Paris (FR)

(72) Inventor: Evan MARGARETTA, Matawan, NJ (US)

(73) Assignee: L'OREAL, Paris (FR)

(21) Appl. No.: 18/678,857

(22) Filed: May 30, 2024

(51) Int. Cl. A61K 8/06; A61Q 19/08

(52) U.S. Cl. CPC A61K 8/066; A61Q 19/08; A61K 2800/412; A61K 2800/54; A61K 2800/594; A61K 2800/652; A61K 2800/95

(57) ABSTRACT

A cosmetic composition that immediately smooths and tightens the skin is described. The cosmetic composition includes an aqueous carrier phase, an oil phase dispersed throughout the aqueous carrier phase, and discrete wax particles separately dispersed throughout the aqueous carrier phase. The aqueous carrier phase includes one or more hydrophilic film forming polymers, one or more water soluble solvent, and water. The oil phase includes one or more hydrophobic film forming polymers and one or more volatile hydrocarbon oils. The wax particles are formed from a wax having a melting point of at least 50° C. The cosmetic compositions are useful in methods for improving the appearance of skin, for example, by smoothing, tightening, and concealing imperfection of the skin.

### FIELD OF THE DISCLOSURE

The present disclosure is drawn to a cosmetic composition that, upon application to skin, immediately smooths and tightens the skin. It also relates to methods for improving the appearance of skin.

### BACKGROUND

Skin acts as a natural barrier between internal and external environments and therefore plays an important role in vital biological functions. It also provides protection against mechanical and chemical injury, microorganisms, and ultraviolet damage. The health and appearance of skin, however, can deteriorate due to environmental factors, genetic makeup, nutrition, and sun exposure. With aging, the outer skin layer (epidermis) thins, even though the number of cell layers remains unchanged. Skin can appear pale and/or develop pigmented spots (age spots, liver spots, or lentigos). Changes in the connective tissue reduce the skin's strength and elasticity. This is known as elastosis. It is more noticeable in sun-exposed areas (solar elastosis).

Skin produces less collagen and elastin as it ages. For example, after the age of twenty, a person (human) produces about 1 percent less collagen in the skin each year. As a result, the skin becomes thinner and more fragile. Inevitably, wrinkles, crow's feet, age-spots, eye bags, and the like, begin to form. In addition, many individuals suffer from scarring or other aberrations in the skin. Consumers often wish to improve the appearance of such age-related skin imperfections, preferably with instantaneous results. Many consumer products and procedures devoted to hiding and reducing wrinkles are available. Some products and procedures are simple and inexpensive, for example, applying makeup, particularly a primer or colored foundation, to cover the skin (and thereby cover and/or fill the wrinkles and provide a smoother look). Far more expensive and drastic procedures, such as surgical face lifts and Botox® injections, are also used to reduce the appearance of wrinkles. However, many consumers either cannot afford, or do not wish, to undergo such drastic cosmetic procedures. There are several lotions and creams that are formulated to hydrate the skin and make it more supple, thereby reducing the appearance of wrinkles. Some of these products contain active ingredients, for example, that help repair and rejuvenate skin over time. Unfortunately, many of these types of products suffer from various drawbacks.

Makeup products are often visible, offer minimal texture benefits, and have no long-lasting effect on the skin. After removal of the makeup, the skin looks the same as before the makeup was applied. Common skin care products can have chronic, acute or both effects on the skin. Hydration and optical effects are common acute benefits, but these benefits quickly wear-off over time. Products that may eventually improve the health of the skin over time have little or no immediate effect on the appearance of skin.

### SUMMARY OF THE DISCLOSURE

The present disclosure relates to a cosmetic composition that provides an immediate skin smoothing and tightening effect, which is dramatic, when applied to skin. Application of the cosmetic composition to the skin improves the appearance of the skin by reducing the presence of wrinkles, eliminating eye bags, obscuring pores, and blurring skin imperfections such as scarring and uneven skin tone. It also provides an overall radiant appearance.

The cosmetic composition includes a unique combination of hydrophobic and hydrophilic film forming polymers that work in concert to provide a quick tightening effect. Volatile hydrocarbon oils from the cosmetic composition evaporate and water from the cosmetic composition hydrates the skin. In addition, the cosmetic composition includes discrete wax particles dispersed throughout the cosmetic composition, which are independent and distinct from separate oil phase dispersed throughout the aqueous carrier phase. The cosmetic composition is not a typical oil-in-water emulsion because it includes three distinct phases: an aqueous phase, which serves as the carrier phase; and oil phase of droplets dispersed throughout the aqueous phase, like a typical oil-in-water emulsion; and a wax phase of discrete wax particles dispersed throughout the aqueous carrier phase. The wax particles are dispersed separate from the oil phase. Thus, the cosmetic composition may be referred to as an “oil/oil-in-water emulsion (O/O/W)” or a “wax/oil-in-water emulsion” considering it has two distinct fatty phases, an oil phase and a wax phase, both independently dispersed throughout the aqueous carrier phase.

The cosmetic composition typically includes:
(a) an aqueous carrier phase comprising:
   (i) one or more hydrophilic film forming polymers;
   (ii) one or more water soluble solvents; and
   (iii) water;
(b) an oil phase dispersed throughout the aqueous carrier phase, wherein the oil phase comprises:
   (i) one or more hydrophobic film forming polymers; and
   (ii) one or more volatile hydrocarbon oils;
(c) one or more emulsifiers;
(d) discrete wax particles dispersed throughout the aqueous carrier phase; and
(e) optionally, one or more soft-focus powders.

Nonlimiting examples of hydrophilic film forming polymers include polyvinylpyrrolidone, polyvinylpyrrolidone copolymers, carboxylic acid or carboxylate-based homopolymers or copolymers, saccharide-based polymers, non-saccharide-based polymers, or combinations thereof. Preferably, at least one of the one or more hydrophilic film forming polymers is selected from polyvinylpyrrolidone (PVP) and vinylpyrrolidone copolymers selected from polyvinylpyrrolidone (PVP)/vinyl acetate copolymer (PVP/VA copolymer), polyvinylpyrrolidone (PVP)/eicosene copolymer, PVP/hexadecene copolymer, VP/methacrylamide/vinyl imidazole copolymer, or combinations thereof

Nonlimiting examples of water-soluble solvents includes glycerin, C₂-C₆ mono-alcohols, polyols, glycols, or a combinations thereof, for example, ethanol, isopropanol, t-butyl alcohol, ethylene glycol, propylene glycol, butylene glycol, pentylene glycol, dipropylene glycol, glycerin, or combinations thereof. In a preferred embodiment, one of the one or more water soluble solvents is ethanol.

Nonlimiting examples of hydrophobic film forming polymers include polyacrylic acids and polyacrylic acid esters, polymethacrylic acid and polymethacrylic acid esters, polyalkylene oxides, such as polyethylene oxide, polyvinylacetates, polyvinylalcohols, polyvinylacetatephthalates (PVAP), polyvinyl acetate copolymers, polycrotonic acids, pullulan, and pullulan compounds such as trialkylsiloxysilylcarbamoyl pullulan compounds where the alkyl group comprises from 1 to 6 carbon atoms, or combinations thereof. In a preferred embodiment, at least one of the one or more hydrophobic film forming polymers is a trialkylsiloxysilylcarbamoyl pullulan compound with an alkyl group comprising from 1 to 6 carbon atoms, preferably trimethylsiloxysilylcarbamoyl pullulan.

Nonlimiting examples of volatile hydrocarbon oils include polyisobutene, isoparaffin, isohexadecane, isododecane, isodecane, isohexane, undecane, tridecane, dodecane, or combinations thereof.

Nonlimiting examples of emulsifiers include polyglycerol-based emulsifiers, sorbitan fatty esters, sugar fatty esters, polyol fatty esters, ethoxylates thereof, or combinations thereof. In various embodiments, the cosmetic composition includes one or more nonionic emulsifiers, one or more polymeric emulsifiers, or a combination thereof. Preferably, at least one of the one or more emulsifiers is a polymeric emulsifier. Nonlimiting examples include poloxamers (ethoxylated sorbitan), alkyl and polyalkyl ethers of poly(ethylene oxide), taurate copolymers, or combinations thereof.

The one or more wax particles are typically formed from waxes having a melting of at least 50° C. This helps ensure the wax particles do not inadvertently melt and coalesce with the oil phase of the cosmetic composition. Nonlimiting examples of waxes include beeswax, hydrogenated myristyl olive esters, hydrogenated stearyl olive esters, rice bran wax, candelilla wax, carnauba wax, Montan wax, Japan wax, spermacetti wax, ozokerite wax, sunflower wax, or combinations thereof. The particles size of the wax particles will vary. Nonetheless, the wax particles typically have a particle size (number-average particle size) of about 1 to about 100 μm. Preferably, the particle size (or number-average particle size) is from about 1 to about 50 μm, more preferably from about 1 to about 20 μm.

The cosmetic composition may include one or more soft-focus powders, one or more pigments, or combinations thereof. Nonlimiting examples of soft-focus powders include talc, mica, titanated mica, alumina, aluminum silicate, silica, fumed silica, silica silylate, polyamide, poly(methyl (meth)acrylate), polyethylene, polymethylsilsesquioxane, nylon-12, cellulose, polylactic acid, or combinations thereof. Pigments can be characterized as organic pigments and inorganic pigments. In a preferred embodiment, the cosmetic composition includes one or more inorganic pigments. Nonlimiting examples of pigments include titanium dioxide, iron oxide, zirconium oxide, zinc oxide, cerium oxide, magnesium oxide, barium sulfate, calcium sulfate, calcium carbonate, magnesium carbonate, talc, mica, kaolin, sericite, white mica, synthetic mica, phlogopite, lepidolite, biotite, lithia mica, silicate, silica acid anhydride, aluminum silicate, magnesium silicate, calcium silicate, barium silicate, strontium silicate, tungstic acid metal salt, hydroxyapatite, vermiculite, haidilite, bentonite, montmorillonite, hectorite, zeolite, ceramic powder, calcium phosphate, dibasic, alumina, aluminum hydroxide, boron nitride, silica, iron oxide red, iron sulfate, black iron oxide, cobalt titanate, crimson mica, or combinations thereof.

The cosmetic composition is particularly useful in methods for improving the appearance of skin, especially skin of the face. The method comprises applying an effective amount of the cosmetic composition to the skin, for example, the face. For example, the cosmetic composition may be applied to the skin using one hands or by using an applicator tool. Upon application of the cosmetic composition to the skin, the composition imparts smoothing and blurring effects to the skin, while immediately providing a tightening effect to the skin. Accordingly, the cosmetic composition is useful in methods for smoothing the skin, methods for tightening the skin, and methods for covering or obscuring imperfection of the skin.

### DETAILED DESCRIPTION OF THE DISCLOSURE

The cosmetic composition provides an immediate smoothing, tightening, and blurring effects upon application to the skin, which is particularly useful for improving the appearance of the skin. The blurring effects masks imperfections and leaves the skin with a uniform and radiant appearance. The tightening effects eliminates wrinkles and eye bags, resulting in a youthful look. The cosmetic compositions may include one or more soft-focus powders, which contribute to the composition's smoothing and blurring effects. In addition, the cosmetic composition includes discrete wax particles, which the inventors found to be surprisingly effective for providing or improving wrinkle reduction, for example, by filling the wrinkle. In addition, the wax particles simultaneously improve the composition's mechanical properties, for example, the composition's tightening, adhesion, and lastingness effects. Instead of disrupting the mechanical properties needed for a successful skin tightening and perfecting composition, inclusion of the discrete wax particles surprisingly enhanced such properties. This is contrary to what was expected.

The cosmetic composition typically includes:
(a) an aqueous carrier phase comprising:
   (i) about 1 to about 8 wt. % of one or more hydrophilic film forming polymers;
   (ii) about 1 to about 25 wt. % of one or more water soluble solvents; and
   (iii) about 40 to about 80 wt. % of water;
(b) an oil phase dispersed throughout the aqueous carrier phase, the oil phase comprising:
   (i) about 1 to about 8 wt. % of one or more hydrophobic film forming polymers; and
   (ii) about 1 to about 15 wt. % of one or more volatile hydrocarbon oils;
(c) about 0.5 to about 5 wt. % of one or more emulsifiers;
(d) about 1 to about 10 wt. % of discrete wax particles; and
(e) optionally, one or more soft-focus powders, one or more pigments, or combinations thereof;
   wherein all weight percentages are based on a total weight of the composition.

As shown above, the cosmetic composition includes both hydrophobic and hydrophilic film forming polymers, in different phases of the composition. These film forming polymers work together to form a durable film on the skin that undergoes an immediate tightening effect upon application to the skin. Upon application to the skin, volatile hydrocarbon oils from the cosmetic composition evaporate as the composition dries. A portion of water from the cosmetic composition also likely evaporates but a portion of the water is available to provide a hydrating effect to the skin.

The cosmetic composition is different from a typical oil-in-water emulsion in which a single oil phase is dispersed throughout a single aqueous carrier phase because the cosmetic composition of the instant case includes discrete wax particles that are independent from the oil phase. Thus, the cosmetic composition includes three distinct phases: (i) an aqueous phase, which serves as the carrier phase; (ii) an oil phase dispersed throughout the aqueous phase typically in the form of oil droplets; and a wax phase consisting of discrete wax particles dispersed throughout the aqueous carrier phase. The oil droplets dispersed throughout the composition and the wax particles dispersed throughout the composition are different from one another. Accordingly, for purposes of the instant disclosure, the cosmetic composition may be referred to as an “oil/oil-in-water emulsion (O/O/W)” or a “wax/oil-in-water” emulsion.

Having the wax particles independently dispersed throughout the aqueous carrier is particularly beneficial. The inventors found that if the wax particles are loaded into the oil phase, the wax particles dissolve in the oil phase, resulting in a single oil phase with no wax particles. An identical composition having the wax particles dissolved in the oil phase becomes too thick—the viscosity cannot accurately be measured nor can the composition be applied to the skin. Having discrete particles dispersed throughout the aqueous carrier phase, independent from the oil phase, allows for a much higher amount of wax (as discrete particles) to be incorporated into the composition, which results in favorable aesthetic and mechanical properties.

### (a) Aqueous Phase

The aqueous phase includes water and ingredients solubilized in the water.

The total amount of aqueous carrier phase in the cosmetic composition will vary but is typically about 55 to about 90 wt. %, based on the total weight of cosmetic composition. In further embodiments, the cosmetic composition includes about 60 to about 90 wt. %, about 65 to about 90 wt. %, about 70 to about 90 wt. %, about 75 to about 90 wt. %, about 55 to about 85 wt. %, about 60 to about 85 wt. %, about 65 to about 85 wt. %, about 70 to about 85 wt. %, about 75 to about 85 wt. % of the aqueous carrier phase, based on the total weight of the cosmetic composition. Preferably, the cosmetic composition includes about 60 to about 90 wt. %, more preferably about 70 to about 90 wt. %, and even more preferably about 75 to about 85 wt. % of aqueous carrier phase, based on the total weight of the cosmetic composition.

### (a) (i) Hydrophilic Film Forming Polymers

A “hydrophilic film forming polymer” is a film-forming polymer that is soluble in water or can be solubilized in water. A “film-forming polymer” is a polymer capable of forming a film upon application to the skin of a human. The one or more hydrophilic film forming polymers, in various embodiments, are preferably nonionic. Nonlimiting examples include polyalkyloxazolines, vinyl acetate homopolymers, vinyl acetate copolymers, acrylic ester homopolymers and copolymers, copolymers of acrylonitrile and of a nonionic monomer, styrene homopolymers, styrene copolymers, copolymers of styrene and of butadiene, or copolymers of styrene, of butadiene and of vinylpyridine, polyamides, vinyllactam homopolymers, vinyllactam copolymers, vinylpyrrolidone/methacrylamide/vinylimidazole terpolymers, dimethicone copolymers, dimethicone crosspolymer, or combinations thereof. In a preferred embodiment, one of the one or more hydrophilic film forming polymers is vinylpyrrolidone/methacrylamide/vinyl imidazole copolymer.

More broadly, in various embodiments, the one or more hydrophilic film forming polymers may be selected from polyvinylpyrrolidone (PVP) polymers, vinylpyrrolidone copolymers, carboxylic acid or carboxylate-based homo- and copolymers, saccharide-based polymers (e.g., polysaccharides), non-saccharide-based polymers, and combinations thereof.

In a preferred embodiments, at least one of the one or more hydrophilic film forming polymers is selected from polyvinylpyrrolidone and the vinylpyrrolidone copolymers. Nonlimiting examples include polyvinylpyrrolidone (PVP), polyvinylpyrrolidone (PVP)/vinyl acetate copolymer (PVP/VA copolymer), polyvinylpyrrolidone (PVP)/eicosene copolymer, PVP/hexadecene copolymer, VP/methacrylamide/vinyl imidazole copolymer. Commercially available polyvinylpyrrolidone includes LUVISKOL® K30, K85, K90 available from BASF. Commercially available copolymers of vinylpyrrolidone and vinylacetate include LUVISKOL® VA37, VA64 available from BASF; copolymers of vinylpyrrolidone, methacrylamide, and vinylimidazole (INCI: VP/methacrylamide/vinyl imidazole copolymer) is commercially available as LUVISET® from BASF.

Nonlimiting examples of carboxylic acid and carboxylic acid/carboxylate-based homopolymers and copolymers include one or more monomers derived from acrylic acid, substituted acrylic acid, salts thereof, or esters thereof (acrylates). Commercially available polymers include those sold under the trade names CARBOPOL®, ACRYSOL®, POLYGEL®, SOKALAN®, CARBOPOL ULTREZ®, and POLYGEL®. Examples of commercially available carboxylic acid polymers include the carbomers, which are homopolymers of acrylic acid crosslinked with allyl ethers of sucrose or pentaerythritol. The carbomers are available as the CARBOPOL® 900 series from B.F. Goodrich (e.g., CARBOPOL® 954). In addition, other suitable carboxylic acid polymeric agents include ULTREZ® 10 (B.F. Goodrich) and copolymers of C10-30 alkyl acrylates with one or more monomers of acrylic acid, methacrylic acid, or one of their short chain (i.e., C1-4 alcohol) esters, wherein the crosslinking agent is an allyl ether of sucrose or pentaeryhtritol. These copolymers are known as acrylates/C10-C30 alkyl acrylate crosspolymers and are commercially available as CARBOPOL® 1342, CARBOPOL® 1382, PEMULEN@ TR-1, and PEMULEN@ TR-2, from B.F. Goodrich.

Other suitable carboxylic acid or carboxylate film forming polymers include copolymers of acrylic acid and alkyl C5-C10 acrylate, copolymers of acrylic acid and maleic anhydride, and polyacrylate crosspolymer-6. Polyacrylate Crosspolymer-6 is amiable in the raw material known as SEPIMAX ZEN® from Seppic.

Another suitable carboxylic acid or carboxylate film forming polymer includes acrylamidopropyltrimonium chloride/acrylates copolymer, a cationic acrylates copolymer (or a quaternary ammonium compound), available as a raw material known under the tradename of SIMULQUAT® HC 305 from Seppic. In certain embodiments, the carboxylic acid or carboxylate film forming polymer is selected from carbomers, acrylates/C₁₀-C₃₀ alkyl acrylate crosspolymers, polyacrylate crosspolymer-6, acrylamidopropyltrimonium chloride/acrylates copolymer, and combinations thereof.

The hydrophilic film forming polymer can be saccharide-based or nonsaccharide-based. The term “saccharide-based” refers to polysaccharides and derivatives thereof. Common derivatives include esters and ethers. Using cellulose as an example, derivatives cellulose include, but are not limited to, cellulose esters, cellulose ethers, methyl cellulose, hydroxypropyl cellulose, hydroxpropylmethyl cellulose, cellulose acetate, and the like. Other polysaccharides can similarly be derivatized. Nonlimiting examples of saccharide-based film forming polymers include alkylcelluloses, hydroxylalkylcelluloses, and hydroxylalkylalkylcelluloses, such as methylcellulose, hydroxylmethylcellulose, hydroxyethylcellulose, hydroxylpropylcellulose (HPC), hydroxyethylmethylcellulose, hydroxypropylmethylcellulose (HPMC) or hypromellose and hydroxybutylmethylcellulose, pullulan, carboxymethylcellulose (CMC) such as sodium CMC, and combinations thereof.

Nonlimiting examples of non-saccharide-based hydrophilic film forming polymers include polyacrylic acids and polyacrylic acid esters, polymethacrylic acid and polymethacrylic acid esters, polyalkylene oxides, such as polyethylene oxide, polyvinylacetates, polyvinylalcohols, polyvinylacetatephthalates (PVAP), polyvinylpyrrolidone (PVP) or povidone, polyvinyl acetate copolymers, and polycrotonic acids.

In a particular embodiments, one or more of the hydrophilic film forming polymer is selected from polyacrylic acids and polyacrylic acid esters, polymethacrylic acid and polymethacrylic acid esters, polyalkylene oxides, such as polyethylene oxide, polyvinylacetates, polyvinylalcohols, polyvinylacetatephthalates (PVAP), polyvinylpyrrolidone (PVP) or povidone, polyvinyl acetate copolymers, polycrotonic acids, or combinations thereof.

The amount of hydrophilic film forming polymer(s) in the cosmetic composition will vary but typically, the hydrophilic film forming polymer(s) are in an amount of about 0.1 to about 12 wt. %, based on the total weight of the cosmetic composition. In further embodiments, the cosmetic composition includes about 0.1 to about 10 wt. %, about 0.1 to about 8 wt. %, about 0.1 to about 5 wt. %, about 0.5 to about 12 wt. %, about 0.5 to about 10 wt. %, about 0.5 to about 8 wt. %, about 0.5 to about 5 wt. %, about 1 to about 12 wt. %, about 1 to about 10 wt. %, about 1 to about 8 wt. %, or about 1 to about 5 wt. %, based on the total weight of the cosmetic composition. Preferably, the cosmetic composition includes about 0.1 to about 10 wt. %, preferably about 0.5 to about 8 wt. %, and more preferably about 1 to about 5 wt. % of the one or more hydrophilic film forming polymers, based on the total weight of the cosmetic composition.

### (a) (ii) Water Soluble Solvent

The term “water soluble solvent” is interchangeable with the term “water soluble organic solvent,” and means an organic solvent that is water soluble or can be solubilized in water and is a liquid at 25° C. and at atmospheric pressure (760 mmHg). For example, water soluble solvents may have a solubility of at least 50% in water under these conditions. Preferably, however, the water-soluble solvent have a solubility of at least 60%, 70%, 80%, or 90%.

Nonlimiting examples of water soluble solvents include glycerin, C₂-C₆ mono-alcohols, polyols, glycols, glycol ethers (for example, monomethyl, monoethyl, and monobutyl ethers of ethylene glycol or propylene glycol), propylene glycol, butylene glycol, hexylene glycol, caprylyl glycol, dipropylene glycol, alkyl ethers of diethylene glycol such as monoethyl ether or monobutyl ether of diethylene glycol (e.g., diethylene glycol monobutyl ether and diethylene glycol monoethyl ether), dipropylene glycol, and alkyl ethers of diethylene glycol, for example monoethyl ether or monobutyl ether of diethylene glycol.

In various embodiment, one or more water soluble solvents are selected from alkanediols (polyhydric alcohols or polyols) such as glycerin, 1,2,6-hexanetriol, trimethylolpropane, ethylene glycol, propylene glycol, diethylene glycol, triethylene glycol, caprylyl glycol, tetraethylene glycol, pentaethylene glycol, dipropylene glycol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, or combinations thereof.

Nonlimiting examples of polyols (polyhydric alcohols) include glycerin, ethylene glycol, diethylene glycol, triethylene glycol, propylene glycol, dipropylene glycol, tripropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, and a combination thereof. Polyol compounds may also be used. Non-limiting examples include the aliphatic diols, such as 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, and 2-ethyl-1,3-hexanediol, of combinations thereof.

The total amount of the one or more water soluble solvents in the cosmetic composition will vary but is typically about 1 to about 25 wt. %, based on the total weight of the cosmetic composition. In further embodiments, the cosmetic composition includes about 1 to about 22 wt. %, about 1 to about 20 wt. %, about 1 to about 18 wt. %, about 1 to about 15 wt. %, about 2 to about 25 wt. %, about 2 to about 22 wt. %, about 2 to about 20 wt. %, about 2 to about 18 wt. %, about 2 to about 15 wt. %, about 4 to about 25 wt. %, about 4 to about 22 wt. %, about 4 to about 20 wt. %, about 4 to about 18 wt. %, about 4 to about 15 wt. %, about 5 to about 25 wt. %, about 5 to about 22 wt. %, about 5 to about 20 wt. %, about 5 to about 18 wt. %, about 5 to about 15 wt. %, about 8 to about 25 wt. %, about 8 to about 22 wt. %, about 8 to about 20 wt. %, or about 8 to about 15 wt. % of one or more water soluble solvents, based on the total weight of the cosmetic composition. Preferably, the cosmetic composition includes about 2 to about 25 wt. %, more preferably about 3 to about 22 wt. %, and even more preferably about 5 to about 15 wt. % of one or more water soluble solvents, based on the total weight of the cosmetic composition.

(a) (iii) Water

The aqueous phase of the cosmetic composition typically includes a large proportion of water. The total amount of water in the cosmetic composition will vary but is typically about 40 to about 85 wt. %, based on the total weight of the cosmetic composition. In further embodiments, the cosmetic composition includes about 45 to about 85 wt. %, about 50 to about 85 wt. %, about 40 to about 80 wt. %, about 45 to about 80 wt. %, about 50 to about 80 wt. %, about 40 to about 75 wt. %, about 45 to about 75 wt. %, about 50 to about 75 wt. %, about 40 to about 70 wt. %, about 45 to about 70 wt. %, about 50 to about 70 wt. %, based on the total weight of the cosmetic composition

### (b) Oil Phase

The oil phase includes the volatile hydrocarbon oil and ingredients solubilized in the volatile hydrocarbon oil.

The total amount of oil phase will vary. Nonetheless, the total amount of oil phase is typically about 3 to about 25 wt. %, based on the total weight of the cosmetic composition. In further embodiments, the cosmetic composition includes about 3 to about 20 wt. %, about 3 to about 18 wt. %, about 3 to about 15 wt. %, about 3 to about 12 wt. %, about 3 to about 10 wt. %, about 5 to about 25 wt. %, about 5 to about 20 wt. %, about 5 to about 18 wt. %, about 5 to about 15 wt. %, about 5 to about 12 wt. %, or about 5 to about 10 wt. %, of the oil phase, based on the total weight of the cosmetic composition. Preferably, the cosmetic composition includes about 3 to about 18 wt. %, more preferably about 5 to about 15 wt. %, and even more preferably about 5 to about 12 wt. % of oil phase, based on the total weight of the cosmetic composition.

### Ratio of Aqueous Carrier Phase to Oil Phase ((a):(b))

The weight ratio of the aqueous carrier phase to the oil phase will vary but it typically about 20:1 to about 4:1 ((a):(b)). The wax particles are not considered part of the oil phase even though waxes are oily/fatty substances because the wax particles form an independent wax particle phase. In various embodiments, the weight ratio of the aqueous carrier phase to the oil phase is about 18:1 to about 4:1, about 16:1 to about 4:1, about 15:1 to about 4:1, about 20:1 to about 5:1, about 18:1 to about 5:1, about 16:1 to about 5:1, or about 15:1 to about 5:1. In further embodiments, the weight ratio of the aqueous carrier phase to the oil phase is about 12:1 to about 4:1, about 10:1 to about 4:1, about 8:1 to about 4:1, about 12:1 to about 5:1, about 10:1 to about 5:1, or about 8:1 to about 5:1. In yet another embodiment, the weight ratio of the aqueous carrier phase to the oil phase is about 20:1 to about 6:1, about 18:1 to about 6:1, about 20:1 to about 8:1, or about 18:1 to about 8:1. Preferably, the weight ratio of the aqueous carrier phase to the oil phase is about 20:1 to about 5:1, more preferably about 18:1 to about 6:1, and even more preferably, about 18:1 to about 6:1.

### (b) (i) Hydrophobic Film Forming Polymer

The term “hydrophobic film-forming polymer” is interchangeable with the term “liposoluble film-forming polymer,” and is a film-forming polymer that is soluble in oils or can be solubilized in oils and forms a film on skin.

Nonlimiting examples include trimethylsiloxysilicate, acrylates/isobornyl acrylate copolymer, acrylic acid/isobutyl acrylate/isobornyl acrylate copolymer, acrylates/stearyl methacrylate copolymer, acrylate/polytrimethyl siloxymethacrylate copolymer, C30-45 alkyldimethylsilylpolypropylsilsequixane, trimethylsilsesquixane, polypropylsilsesquixane, acrylates/dimethicone copolymer, octylacrylamide/acrylates/butylaminoethyl methacrylate copolymer, VA/butyl maleate/isobornyl acrylate copolymer, acrylates/t-butylacrylamide copolymer, polyvinylpyrrolidone/vinyl acetate copolymer, triacontanyl PVP copolymer, acrylates/dimethylaminoethyl methacrylate copolymer, norbornene/tris(trimethylsiloxy) silylnorbornenecopolymer, styrene/butadiene copolymer and combinations thereof.

The hydrophobic film-forming polymer can be of natural or synthetic origin, and in various embodiments, are selected from trimethylsiloxysilicates, in particular Belsil® TMS 803 (WACKER®), KF7312 from Shin Etsu®; phenylalkylsiloxysilicates, in which the alkyl group preferably comprises 1 to 6 carbon atoms, such as phenylpropyldimethylsiloxysilicate; silicone acrylate polymers such as acrylate/dimethicone copolymers, and in particular acrylate/dimethicone copolymers (for example, KP-545 from Shin-Etsu®); acrylate/dimethicone copolymers in (for example KP-549 and KP-579 from Shin-Etsu®), and acrylate/dimethicone copolymers in isododecane (such as, for example, KP-550 from Shin-Etsu®); acrylate/polytrimethylsiloxymethacrylate copolymers and, in particular, acrylate/polytrimethylsiloxymethacrylate copolymers in dimethicone (such as, for example, FA-4003 DM from Dow Corning®); acrylate/polytrimethylsiloxymethacrylate copolymers in isododecane (such as, for example, FA-4004 ID from Dow Corning®); polyalkylsilsesquioxanes comprising 1 to 6 carbon atoms, and preferably polymethylsilsesquioxane (such as, for example, Silform® Flexible Resin from Momentive®); trialkylsiloxysilylcarbamoyl pullulans, in which the alkyl group comprises 1 to 6 carbon atoms, and preferably trimethylsiloxysilylcarbamoyl pullulan (such as, for example, TSPL-30-ID from Shin-Etsu®); copolymers of vinylpyrrolidone (VP) and alkene comprising 2 to 20 carbon atoms, such as VP/eicosene, VP/hexadecene and VP/styrene copolymers; copolymers of a vinyl ester, and preferably vinyl acetate/allyl stearate, vinyl acetate/vinyl laurate, vinyl acetate/vinyl stearate, vinyl acetate/vinyl octadecene, vinyl acetate/octadecyl vinyl ether, vinyl propionate/allyl laurate, vinyl propionate/vinyl laurate, vinyl stearate/octadecene-1, vinyl acetate/dodecene-1, vinyl stearate/ethyl vinyl ether, vinyl propionate/cetyl vinyl ether, vinyl stearate/allyl acetate, vinyl dimethyl-2,2-octanoate/vinyl laurate, allyl dimethyl-2,2-pentanoate/vinyl laurate, vinyl dimethyl propionate/vinyl stearate, and allyl dimethyl propionate/vinyl stearate copolymers; hydrogenated or non-hydrogenated polyolefins and preferably polymers or copolymers of alkenes comprising 2 to 20 carbon atoms, such as the polybutenes, polyisobutenes and polydecenes; or combinations thereof.

In a preferred embodiment, at least one of the one or more hydrophobic film forming polymers is a silicone film-forming polymer. Nonlimiting examples include trimethylsiloxysilicates; phenylalkylsiloxysilicates, in which the alkyl group preferably comprises 1 to 6 carbon atoms, such as phenylpropyldimethylsiloxysilicate; silicone acrylate polymers such as acrylate/dimethicone copolymers, and in particular acrylate/dimethicone copolymers, and acrylate/dimethicone copolymers, acrylate/polytrimethylsiloxymethacrylate copolymers and, in particular, acrylate/polytrimethylsiloxymethacrylate copolymers, acrylate/polytrimethylsiloxymethacrylate copolymers, polyalkylsilsesquioxanes comprising 1 to 6 carbon atoms, and preferably polymethylsilsesquioxane trialkylsiloxysilylcarbamoyl pullulans, in which the alkyl group comprises 1 to 6 carbon atoms, and preferably trimethylsiloxysilylcarbamoyl pullulan (such as, for example, TSPL-30-ID from Shin-Etsu®).

Particularly preferred hydrophobic film forming polymers are trialkylsiloxysilylcarbamoyl pullulan compounds with alkyl groups comprising from 1 to 6 carbon atoms. These alkyl groups can be linear or branched. A particularly preferred hydrophobic film forming polymer is trimethylsiloxysilylcarbamoyl pullulan.

The total amount of the one or more hydrophobic film forming polymers in the cosmetic composition will vary but is typically from 0.1 to about 10 wt. %, based on the total weight of the cosmetic composition. In further embodiments, the cosmetic composition includes about 0.1 to about 8 wt. %, about 0.1 to about 5 wt. %, about 0.1 to about 3 wt. %, about 0.5 to about 10 wt. %, about 0.5 to about 8 wt. %, about 0.5 to about 5 wt. %, about 1 to about 10 wt. %, about 1 to about 8 wt. %, about 1 to about 5 wt. %, about 1 to about 3 wt. %, about 1.5 to about 10 wt. %, about 1.5 to about 8 wt. %, about 1.5 to about 5 wt. %, about 1.5 to about 3 wt. %, about 2 to about 10 wt. %, about 2 to about 8 wt. %, or about 2 to about 5 wt. % of the one or more hydrophobic film forming polymers, based on the total weight of the cosmetic composition. Preferably, the cosmetic composition includes about 0.5 to about 8 wt. %, more preferably about 0.5 to about 5 wt. %, and even more preferably, 1 to about 3 wt. % of one or more hydrophobic film forming polymers, based on the total weight of the cosmetic composition.

### Weight Ratio of Hydrophilic Film Forming Polymer to Hydrophobic Film Forming Polymer ((a) (i):(b) (i))

The weight ratio of the hydrophilic film forming polymer to the hydrophobic film forming polymer will vary but is typically about 5:1 to about 1:5 ((a) (i):(b) (i)). In further embodiments, the weight ratio of (a) (i) to (b) (i) is about 4:1 to about 1:4, preferably about 3:1 to about 1:3, or more preferably about 2:1 to about 1:2.

### (b) (ii) Volatile Hydrocarbon Oil

The term “volatile hydrocarbon oil” is a hydrocarbon that is volatile at ambient temperature (25° C.) and normal pressure (1 atm). The term “oil” is understood to mean a fatty compound that is liquid at ambient temperature (25° C.) and normal pressure (1 atm); and when added in an amount of at least 1% by weight into water at 25° C., the oil is not soluble in the water or only soluble to a level of 10% or less by weight, with respect to the weight of the oil added into the water. The term “hydrocarbon oil” is oil comprising hydrogen and carbon atoms, which does not contain silicon atoms.

Suitable volatile hydrocarbon oils include, but are not limited to, those having from 8 to 16 carbon atoms and their combinations and in particular branched C₈ to C₁₆ alkanes such as C₈ to C₁₆ isoalkanes (also known as isoparaffins), isododecane, isodecane, isohexadecane, and for example, the oils sold under the trade names of ISOPAR or PERMETHYL, the C₈ to C₁₆ branched esters such as isohexyl or isodecyl neopentanoate and their combinations. Preferably, the volatile hydrocarbon oils have a flash point of below 60° C.

Nonlimiting examples of volatile hydrocarbon oils include isoparaffin, isohexadecane, isododecane, isodecane, isohexane, undecane, tridecane, dodecane, C₁₃-C₁₄ isoalkane, or combinations thereof. Preferably, at least one of the one or more volatile hydrocarbon oils isododecane, isohexadecane, C₁₃-C₁₄ isoalkane, or combinations thereof.

The total amount of the volatile hydrocarbon oil in the cosmetic composition will vary but is typically about 1 to about 20 wt. %, based on the total weight of the cosmetic composition. In further embodiments, the cosmetic composition includes about 1 to about 18 wt. %, about 1 to about 15 wt. %, about 1 to about 12 wt. %, about 1 to about 10 wt. %, about 2 to about 20 wt. %, about 2 to about 18 wt. %, about 2 to about 15 wt. %, about 2 to about 12 wt. %, about 2 to about 10 wt. %, about 3 to about 20 wt. %, about 3 to about 18 wt. %, about 3 to about 15 wt. %, about 3 to about 12 wt. %, or about 3 to about 10 wt. %, based on the total weight of the cosmetic composition. Preferably, the cosmetic composition includes about 2 to about 18 wt. %, more preferably about 2 to about 15 wt. %, and even more preferably, about 3 to about 10 wt. % of the one or more volatile hydrocarbon oils, based on the total weight of the cosmetic composition.

Weight Ratio of Hydrophobic Film Forming Polymer to Volatile Hydrocarbon Oil ((b) (i):(b) (ii))

The weight ratio of the one or more hydrophobic film forming polymers to the one or more volatile hydrocarbon oils will vary but is typically about 1:1 to about 1:10 ((b) (i):(b) (ii)). In further embodiments, the weight ratio of (b) (i) to (b) (ii) is about 1:1 to about 1:8, preferably about 1:1.5 to about 1:6, more preferably about 1:2 to about 1:6.

### (c) Emulsifiers

The cosmetic composition includes one or more emulsifiers. The emulsifiers include polymeric emulsifiers and non-polymeric emulsifiers. Furthermore, the emulsifiers may be nonionic, anionic, amphoteric, or a combination thereof. Cationic emulsifiers may also be useful but generally, nonionic, and anionic emulsifiers are preferred.

In a preferred embodiment, the cosmetic composition includes one or more polymeric emulsifiers, one or more nonionic emulsifiers, or a combination thereof.

Nonlimiting examples of polymeric emulsifiers include poloxamers, alkoxylated surfactants, polyacrylamide, acryloyldimethyltaurate polymers, or combinations thereof. As the name indicates, polymeric emulsifiers provide emulsifying properties to the cosmetic composition. Nonetheless, in some cases, in addition to their emulsifying properties, the polymeric emulsifiers have additional influence other characteristics of the cosmetic composition, for example, the polymeric emulsifiers can thicken the cosmetic composition, i.e., behave as a thickening agent by increasing the viscosity of the cosmetic composition. In some cases, a polymeric emulsifier may have film forming properties in addition to its emulsifying properties. In instances where a polymeric emulsifier also provides thickening or behaves as a film forming polymer, a single compound (a single polymer/copolymer) will serve as only a polymeric emulsifier or film-forming polymer. A single compound (a single polymer/copolymer) cannot simultaneously serve as both the polymeric emulsifier and the film-forming polymer when the cosmetic composition includes both a polymeric emulsifier and a film-forming polymer.

Poloxamers are nonionic triblock copolymers composed of a central hydrophobic chain of polyoxypropylene (poly(propylene oxide)) flanked by two hydrophilic chains of polyoxyethylene (poly(ethylene oxide)). The term poloxamer emulsifiers refers to a polyethylene oxide-polypropylene oxide-polyethylene oxide (PEG-PPG-PEG) nonionic triblock copolymer. Poloxamer emulsifiers include nonionic triblock copolymers such as polyoxyethylene oxide-polyoxypropylene oxide-polyoxyethylene oxide (PEO-PPO-PEO) characterized by a central hydrophobic chain of polyoxypropylene (poly(propylene oxide)) flanked by two hydrophilic chains of polyoxyethylene (poly(ethylene oxide)). Exemplary nonionic triblock copolymers may comprise a structure according to Formula (A):

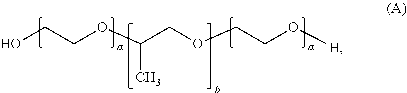

wherein each a is independently an integer in the range of 2-130, and b is an integer in the range of 15-67. In some embodiments, a is in the range of 50-100 and b is in the range of 20-40. In some embodiments, a is in the range of 70-90 and b is in the range of 25-30. Nonlimiting examples of poloxamers include poloxamer 188, also known as Pluronic® F-68, or KOLLIPHOR® P188, e.g., having a=80 and b=27. Other poloxamers include poloxamer 338, also known as Synperonic® PE/F108, poloxamer 407, also known as Synperonic® PE/F127, poloxamer 331, also known as Synperonic® PE/L101.

The term “PLURONIC® F68,” “Pluronic F-68”, or “PF-68”, also known as poloxamer 188, refers to poly(ethylene glycol)-block-poly(propylene glycol)-block poly(ethylene glycol) copolymer with an average molecular weight, avg. Mn, of 8350-8400. The term “PLURONIC® F127” also known as poloxamer 407 refers to a triblock copolymer consisting of a central hydrophobic block of polypropylene glycol flanked by two hydrophilic blocks of polyethylene glycol (PEG). The approximate lengths of the two PEG blocks is 101 repeat units, while the approximate length of the propylene glycol block is 56 repeat units. This is also known by the Croda trade name Synperonic® PE/F 127. The term “PLURONIC® F108” refers to poly(ethylene glycol)-block-poly(propylene glycol)-block poly(ethylene glycol). The term “PLURONIC® P103” refers to poly(ethylene glycol)-block-poly(propylene glycol)-block poly(ethylene glycol). The term “PLURONIC® P104” refers to poly(ethylene glycol)-block-poly(propylene glycol)-block poly(ethylene glycol). The term “PLURONIC® P123” refers to poly(ethylene glycol)-block-poly(propylene glycol)-block poly(ethylene glycol).

Nonlimiting examples of poloxamers include poloxamer-101, poloxamer-105, poloxamer-105 benzoate, poloxamer-108, poloxamer-122, poloxamer-123, poloxamer-124, poloxamer-181, poloxamer-182, poloxamer-182 dibenzoate, poloxamer-183, poloxamer-184, poloxamer-185, poloxamer-188, poloxamer-212, poloxamer-215, poloxamer-217, poloxamer-231, poloxamer-234, poloxamer-235, poloxamer-237, poloxamer-238, poloxamer-282, poloxamer-284, poloxamer-288, poloxamer-331, poloxamer-333, poloxamer-334, poloxamer-335, poloxamer-338, poloxamer-401, poloxamer-402, poloxamer-403, and poloxamer-407.

The lengths of the polymer blocks can be customized and therefore, many different poloxamers exist that have slightly different properties. Poloxamer copolymers are commonly named with the letter “P” (for poloxamer) followed by three digits, the first two digits×100 give the approximate molecular mass of the polyoxypropylene core, and the last digit×10 gives the percentage polyoxyethylene content (e.g., P407=Poloxamer with a polyoxypropylene molecular mass of 4,000 g/mol and a 70% polyoxyethylene content). For the Pluronic® and Synperonic® poloxamer tradenames, coding of these copolymers starts with a letter to define its physical form at room temperature (L=liquid, P=paste, F=flake (solid)) followed by two or three digits. The first digit (two digits in a three-digit number) in the numerical designation, multiplied by 300, indicates the approximate molecular weight of the hydrophobic chain; and the last digit×10 gives the percentage polyoxyethylene content (e.g., F-68 indicates a polyoxypropylene molecular mass of 1,800 g/mol and a 80% polyoxyethylene content). An exemplary poloxamer is Pluronic F-68®. PF-68 is a nonionic triblock copolymer polyoxyethylene oxide-polyoxypropylene oxide-polyoxyethylene oxide (PEO—PPO-PEO). Commercially available poloxamers include PLURONIC® emulsifiers such as PLURONIC® F68, F77, F87, F98, F108, F127, P103, P104, P105, and P123.

Alkoxylated emulsifiers include polyoxyethylenated C8-C30 fatty acid esters (preferably C12-C18) of sorbitan, polyethoxylated C8-C30 (preferably C12-18) fatty alcohols, polyglycerolated C8-C30 (preferably C12-C18) fatty acid esters, polyoxyethylenated compounds having preferably from 2 to 30 moles of ethylene oxide, polyglycerolated compounds having preferably from 2 to 16 moles of glycerol; and combinations thereof. The polyoxyethylenated C8-C30 fatty alcohols may be chosen from C12-C18 fatty alcohols, in particular polyoxyethylenated lauryl alcohol, cetyl alcohol, myristyl alcohol, and stearyl alcohol having from 2 to 30 mol of ethylene oxide, such as: cetyl alcohol polyoxyethylenated with 2 EO (Ceteth-2) (HLB 5.3) cetyl alcohol polyoxyethylenated with 6 EO (Ceteth-6) (HLB 11.1) cetyl alcohol polyoxyethylenated with 10 EO (Ceteth-10) (HLB 12.9) cetyl alcohol polyoxyethylenated with 20 EO (Ceteth-20) (HLB 15.7) cetyl alcohol polyoxyethylenated with 24 EO (Ceteth-24) (HLB 16.3) lauryl alcohol polyoxyethylenated with 2 EO (laureth-2) (HLB 6.1) lauryl alcohol polyoxyethylenated with 3 EO (laureth-3) (HLB 8) lauryl alcohol polyoxyethylenated with 4 EO (laureth-4) (HLB 9.4) lauryl alcohol polyoxyethylenated with 7 EO (laureth-7) (HLB 12.3) lauryl alcohol polyoxyethylenated with 9 EO (laureth-9) (HLB 13.6) lauryl alcohol polyoxyethylenated with 10 EO (laureth-10) (HLB 13.9) lauryl alcohol polyoxyethylenated with 12 EO (laureth-12) (HLB 14.6) lauryl alcohol polyoxyethylenated with 21 EO (laureth-21) (HLB 15.5) lauryl alcohol polyoxyethylenated with 23 EO (laureth-23) (HLB 16.3) stearyl alcohol polyoxyethylenated with 2 EO (Steareth-2) (HLB 4.9) stearyl alcohol polyoxyethylenated with 10 EO (Steareth-10) (HLB 12.4) stearyl alcohol polyoxyethylenated with 20 EO (Steareth-20) (HLB 15.2) stearyl alcohol polyoxyethylenated with 21 EO (Steareth-21) (HLB 15.5).

The polyoxyethylenated C8-C30 fatty acid esters (preferably C12-C18) of sorbitan may be chosen from polyoxyethylenated esters of C12-C18 fatty acids, in particular lauric, myristic, cetylic or stearic acids, of sorbitan especially containing from 2 to 30 mol of ethylene oxide, such as: polyoxyethylenated sorbitan monolaurate (4 EO) (Polysorbate-21) (HLB 13.3) polyoxyethylenated sorbitan monolaurate (20 EO) (Polysorbate-20) (HLB 16.7) polyoxyethylenated sorbitan monopalmitate (20 EO) (Polysorbate-40) (HLB 15.6) polyoxyethylenated sorbitan monostearate (20 EO) (Polysorbate-60) (HLB 14.9) polyoxyethylenated sorbitan monostearate (4 EO) (Polysorbate-61) (HLB 9.6) polyoxyethylenated sorbitan monooleate (20 EO) (Polysorbate-80) (HLB 15). In a preferred embodiment, the cosmetic composition includes one or more nonionic emulsifiers chosen from polyoxyethylenated C8-C30 fatty acid esters (preferably C12-C18) of sorbitan, preferably polyoxyethylenated esters of C12-C18 fatty acids.

The polyglycerolated C8-C30 fatty acid esters may be chosen from polyglycerolated esters of C12-C18 fatty acids, in particular lauric, myristic, palmitic, stearic or isostearic acid, having from 2 to 16 mol of glycerol, such as: polyglyceryl-2 laurate, polyglyceryl-3 laurate, polyglyceryl-4 laurate, polyglyceryl-5 laurate, polyglyceryl-6 laurate, polyglyceryl-10 laurate; polyglyceryl-2 myristate, polyglyceryl-3 myristate, polyglyceryl-4 myristate, polyglyceryl-5 myristate, polyglyceryl-6 myristate, polyglyceryl-10 myristate; polyglyceryl-2 palmitate, polyglyceryl-3 palmitate, polyglyceryl-6 palmitate, polyglyceryl-10 palmitate; polyglyceryl-2 isostearate, polyglyceryl-3 isostearate, polyglyceryl-4 isostearate, polyglyceryl-5 isostearate, polyglyceryl-6 isostearate, polyglyceryl-10 isostearate; polyglyceryl-2 stearate, polyglyceryl-3 stearate, polyglyceryl-4 stearate, polyglyceryl-5 stearate, polyglyceryl-6 stearate, polyglyceryl-8 stearate, polyglyceryl-10 stearate, and combinations thereof.

In a preferred embodiment, the polymeric emulsifier is based on an acrylamido methylpropanesulfonic acid (AMPS) and at least one ethylenically unsaturated monomer comprising at least one hydrophobic part containing from 8 to 30 carbon atoms, in particular from 10 to 30 carbon atoms and more particular from 12 to 22 carbon atoms, in addition, the hydrophobic radical may also comprise at least one alkylene oxide unit and in particular a polyoxyalkylene chain. The polyoxyalkylene chain may be formed from ethylene oxide units and/or propylene oxide units and even more particular be formed solely from ethylene oxide units, preferably, the at least one ethylenically unsaturated monomer is acrylic or methacrylic ester of ethoxylated alcohols RO—(CH2CH2O)mH, in which R is an alkyl radical having 12 to 30 carbon atoms and m is a number from 3 to 35, and CH2-CH—COO—(CH2CH2-COO) nX in which n is a number from 0 to 10 and X is a counterion and is preferably H+, Na+ and/or NH4+. In the case of the copolymers, preferably one or more structural units based on the following comonomers are present in the copolymers: acrylic acid, methacrylic acid, acrylamide, dimethyl-acrylamide, vinylpyrrolidone (VP), hydroxyethyl acrylate, and hydroxyethyl methacrylate. More particularly, the suitable polymeric emulsifier can be copolymer of AMPS and (meth)acrylic ester of ethoxylated C8-C30, or C10-C30, or C12-C22, or C12-C18 fatty alcohol. In some embodiments, the suitable associative polymer can be copolymer of AMPS and (meth)acrylic ester of ethoxylated C8-C30, or C12-C30, or C12-C22, or C12-C18 fatty alcohol, wherein the ethoxylated fatty alcohol in each case comprise 5 to 35, in particular 7 to 30, more in particular 7 to 25 ethylene oxide (EO) radicals. Suitable AMPS copolymers are, for example, ammonium acryloyldimethyltaurate/beheneth-25-methacrylate copolymer such as Aristoflex® HMB from Clariant, ammonium acryloyldimethyltaurate/beheneth-25 methacrylate crosspolymer such as Aristoflex® BLV from Clariant, ammonium Acryloyldimethyltaurate/Steareth-25 Methacrylate crosspolymer such as Aristoflex HMS® by Clariant, ammonium acryloyldimethyltaurate/steareth-8 methacrylate copolymer such as Aristoflex® SNC, Ammonium acryloyldimethyltaurate/laureth-7 methacrylate copolymer such as Aristoflex® LNC.

In a particularly preferred embodiment at least one polymeric emulsifiers is selected from polyacrylamide and acryloyldimethyltaurate polymers such as acrylamide/sodium acryloyldimethyltaurate copolymer (e.g. Sepineo® P600), acrylamide/sodium acryloyldimethyltaurate/acrylic acid copolymer, ammonium acryloyldimethyltaurate/Beheneth-25 methacrylate crosspolymer, ammonium acryloyldimethyltaurate/carboxyethyl acrylate crosspolymer, ammonium acryloyldimethyltaurate/Laureth-7 methacrylate copolymer, ammonium acryloyldimethyltaurate/Steareth-25 methacrylate crosspolymer, ammonium acryloyldimethyltaurate/Steareth-8 methacrylate copolymer, ammonium acryloyldimethyltaurate/vinyl formamide copolymer, ammonium acryloyldimethyltaurate/VP copolymer, ammonium polyacryloyldimethyl taurate, dimethylacrylamide/sodium acryloyldimethyltaurate crosspolymer, HEA/sodium scryloyldimethyltaurate/Steareth-20 methacrylate copolymer, hydroxyethyl acrylate/sodium acryloyldimethyl taurate copolymer, sodium acrylate/acryloyldimethyltaurate/dimethylacrylamide crosspolymer, sodium acrylate/sodium acryloyldimethyl taurate copolymer, sodium acrylate/sodium acryloyldimethyl taurate/acrylamide copolymer, sodium acryloyl dimethyl taurate/PEG-8 diacrylate crosspolymer, sodium acryloyldimethyl taurate/acrylamide/VP copolymer, sodium acryloyl dimethyltaurate/methacrylamidolauric acid copolymer, sodium acryloyldimethyltaurate/VP crosspolymer, sodium polyacryloyldimethyl taurate, or combinations thereof. Particularly preferred polymeric emulsifiers include polyacrylamide, acrylamide/sodium acryloyldimethyltaurate copolymer, or a combination thereof.

Nonlimiting examples of nonionic emulsifiers include poloxamer emulsifiers; polysorbates; alkyl polyglucosides; alcohols, alpha-diols, alkylphenols and esters of fatty acids, being ethoxylated, propoxylated or glycerolated (polyglyceryl-2 isostearate); ethoxylated fatty esters; glyceryl esters of fatty acids; fatty alcohol ethoxylates; alkyl phenol ethoxylates; fatty acid alkoxylates; and combinations thereof.

In various embodiments, the nonionic emulsifiers are selected from alkyl and polyalkyl esters of poly(ethylene oxide), alkyl and polyalkyl ethers of poly(ethylene oxide), optionally polyoxyethylenated alkyl and polyalkyl esters of sorbitan, optionally polyoxyethylenated alkyl and polyalkyl ethers of sorbitan, alkyl and polyalkyl glycosides or polyglycosides, in particular alkyl and polyalkyl glucosides or polyglucosides, alkyl and polyalkyl esters of sucrose, optionally polyoxyethylenated alkyl and polyalkyl esters of glycerol, and optionally polyoxyethylenated alkyl and polyalkyl ethers of glycerol, and combinations thereof. Preferably, the nonionic emulsifiers may be chosen from alkyl and polyalkyl esters of poly(ethylene oxide), alkyl and polyalkyl ethers of poly(ethylene oxide), optionally polyoxyethylenated alkyl and polyalkyl esters of sorbitan, optionally polyoxyethylenated alkyl and polyalkyl ethers of sorbitan, optionally polyoxyethylenated alkyl and polyalkyl esters of glycerol, and optionally polyoxyethylenated alkyl and polyalkyl ethers of glycerol, and combinations thereof.

Nonlimiting examples of sorbitan fatty acid esters include sorbitan isostearate and sorbitan oleate.

In a preferred embodiment, at least one of the one or more nonionic emulsifiers is a sorbitan emulsifier, for example, sorbitan caprylate, sorbitan cocoate, sorbitan diisostearate, sorbitan dioleate, sorbitan distearate, sorbitan isostearate, sorbitan laurate, sorbitan oleate, sorbitan olivate, sorbitan palmitate, sorbitan palmate, sorbitan sesquicaprylate, sorbitan sesquiisostearate, sorbitan sesquioleate, sorbitan sesquistearate, sorbitan stearate, PEG-20 sorbitan cocoate, PEG-40 sorbitan diisostearate, PEG-2 sorbitan isostearate, PEG-5 sorbitan isostearate, PEG-20 sorbitan isostearate, PEG-40 sorbitan laurate, PEG-10 sorbitan laurate, PEG-44 sorbitan laurate, PEG-75 sorbitan laurate, PEG-80 sorbitan laurate, PEG-3 sorbitan oleate, PEG-6 sorbitan oleate, PEG-20 sorbitan oleate, PEG-40 sorbitan oleate, PEG-80 sorbitan palmitate, PEG-3 sorbitan stearate, PEG-4 sorbitan stearate, PEG-6 sorbitan stearate, PEG-40 sorbitan stearate, PEG-60 sorbitan stearate, Polysorbate 20, Polysorbate 21, Polysorbate 40, Polysorbate 60, Polysorbate 61, Polysorbate 65, Polysorbate 80, Polysorbate 81, and Polysorbate 85.

Nonlimiting examples of alkyl and polyalkyl esters of poly(ethylene oxide) include those containing at least one C8-C30 alkyl radical, with a number of ethylene oxide (EO) units ranging from 2 to 200. Mention may be made, for example, of (INCI name) PEG-20 stearate, PEG-40 stearate, PEG-100 stearate, PEG-20 laurate, PEG-8 laurate, PEG-40 laurate, PEG-150 distearate, PEG-7 cocoate, PEG-9 cococate, PEG-8 oleate, PEG-10 oleate and PEG-40 hydrogenated castor oil.

Nonlimiting examples of alkyl and polyalkyl ethers of poly(ethylene oxide) include those containing at least one C8-C30 alkyl radical, with a number of ethylene oxide (EO) units ranging from 3 to 200. Mention may be made, for example, of laureth-3, laureth-4, laureth-7, laureth-23, ceteth-5, ceteth-7, ceteth-15, ceteth-23, oleth-5, oleth-7, oleth-10, oleth-12, oleth-20, oleth-50, phytosterol 30 EO, steareth-6, steareth-20, steareth-21, steareth-40, steareth-100, beheneth 100, ceteareth-7, ceteareth-10, ceteareth-15, ceteareth-25, pareth-3, pareth-23, C12-15 pareth-3, C12-13 pareth-4, C12-13 pareth-23, trideceth-3, trideceth-4, trideceth-5, trideceth-6, trideceth-7 and trideceth-10, and combinations thereof.

Nonlimiting examples of polyoxyethylenated alkyl and polyalkyl esters of sorbitan include those with a number of ethylene oxide (EO) units ranging from 0 to 100. Mention may be made, for example, of sorbitan laurate, sorbitan laurate 4 EO, sorbitan laurate 20 EO (polysorbate 20), sorbitan palmitate 20 EO (polysorbate 40), sorbitan stearate 20 EO (polysorbate 60), sorbitan oleate 20 EO (polysorbate 80) and sorbitan trioleate 20 EO (polysorbate 85). Polyoxyethylenated alkyl and polyalkyl ethers of sorbitan that are preferably used are those with a number of ethylene oxide (EO) units ranging from 0 to 100.

The cosmetic composition may include one or more alkanolamides. Nonlimiting examples alkanolamides include fatty acid alkanolamides. The fatty acid alkanolamides may be fatty acid monoalkanolamides or fatty acid dialkanolamides or fatty acid isoalkanolamides, and may have a C₂₋₈ hydroxyalkyl group (the C₂₋₈ chain can be substituted with one or more than one-OH group). Non-limiting examples include fatty acid diethanolamides (DEA) or fatty acid monoethanolamides (MEA), fatty acid monoisopropanolamides (MIPA), fatty acid diisopropanolamides (DIPA), and fatty acid glucamides (acyl glucamides). Nonlimiting examples of fatty alkanolamides include those formed by reacting an alkanolamine and a C6-C36 fatty acid, for example: oleic acid diethanolamide, myristic acid monoethanolamide, soya fatty acids diethanolamide, stearic acid ethanolamide, oleic acid monoisopropanolamide, linoleic acid diethanolamide, stearic acid monoethanolamide (Stearamide MEA), behenic acid monoethanolamide, isostearic acid monoisopropanolamide (isostearamide MIPA), erucic acid diethanolamide, ricinoleic acid monoethanolamide, coconut fatty acid monoisopropanolamide (cocoamide MIPA), coconut acid monoethanolamide (Cocamide MEA), palm kernel fatty acid diethanolamide, coconut fatty acid diethanolamide, lauric diethanolamide, polyoxyethylene coconut fatty acid monoethanolamide, coconut fatty acid monoethanolamide, lauric monoethanolamide, lauric acid monoisopropanolamide (lauramide MIPA), myristic acid monoisopropanolamide (Myristamide MIPA), coconut fatty acid diisopropanolamide (cocamide DIPA), and combinations thereof. In some instances, the fatty acid alkanolamides may preferably be selected from cocamide MIPA, cocamide DEA, cocamide MEA, cocamide DIPA, and combinations thereof.

The one or more nonionic emulsifiers may include one or more acyl glucamides, for example, acyl glucamides having a carbon chain length of 8 to 20. Nonlimiting examples include lauroyl/myristoyl methyl glucamide, capryloyl/capryl methyl glucamide, lauroyl methyl glucamide, myristoyl methyl glucamide, capryloyl methyl glucamide, capryl methyl glucamide, cocoyl methyl glucamide, capryloyl/caproyl methyl glucamide, cocoyl methyl glucamide, lauryl methylglucamide, oleoyl methylglucamide oleate, stearoyl methylglucamide stearate, sunfloweroyl methylglucamide, and tocopheryl succinate methylglucamide.

The cosmetic composition may include one or more alkyl polyglucosides. Non-limiting examples of alkyl polyglucosides include those having the following formula:

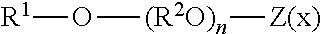

wherein R¹ is an alkyl group having 8-18 carbon atoms;
R² is an ethylene or propylene group;
Z is a saccharide group with 5 to 6 carbon atoms;
n is an integer from 0 to 10; and
x is an integer from 1 to 5.

Preferred alkyl polyglucosides include lauryl glucoside, octyl glucoside, decyl glucoside, coco glucoside, caprylyl/capryl glucoside, and sodium lauryl glucose carboxylate. Typically, the at least one alkyl poly glucoside compound is selected from the group consisting of lauryl glucoside, decyl glucoside coco glucoside, or combinations thereof.

In various embodiments, the nonionic emulsifiers may be selected from esters of polyols with fatty acids with a saturated or unsaturated chain containing for example from 8 to 24 carbon atoms, preferably 12 to 22 carbon atoms, and alkoxylated derivatives thereof, preferably with a number of alkyleneoxide of from 10 to 200, and more preferably from 10 to 100, such as glyceryl esters of a C₈-C₂₄, preferably C₁₂-C₂₂, fatty acid or acids and alkoxylated derivatives thereof, preferably with a number of alkyleneoxide of from 10 to 200, and more preferably from 10 to 100; polyethylene glycol esters of a C₈-C₂₄, preferably C₁₂-C₂₂, fatty acid or acids and alkoxylated derivatives thereof, preferably with a number of alkyleneoxide of from 10 to 200, and more preferably from 10 to 100; sorbitol esters of a C₈-C₂₄, preferably C₁₂-C₂₂, fatty acid or acids and alkoxylated derivatives thereof, preferably with a number of alkyleneoxide of from 10 to 200, and more preferably from 10 to 100; sugar (sucrose, glucose, alkylglycose) esters of a C₈-C₂₄, preferably C₁₂-C₂₂, fatty acid or acids and alkoxylated derivatives thereof, preferably with a number of alkyleneoxide of from 10 to 200, and more preferably from 10 to 100; ethers of fatty alcohols; ethers of sugar and a C₈-C₂₄, preferably C₁₂-C₂₂, fatty alcohol or alcohols; and combinations thereof.

Examples of ethoxylated fatty esters that may be mentioned include the adducts of ethylene oxide with esters of lauric acid, palmitic acid, stearic acid or behenic acid, and combinations thereof, especially those containing from 9 to 100 oxyethylene groups, such as PEG-9 to PEG-50 laurate (as the CTFA names: PEG-9 laurate to PEG-50 laurate); PEG-9 to PEG-50 palmitate (as the CTFA names: PEG-9 palmitate to PEG-50 palmitate); PEG-9 to PEG-50 stearate (as the CTFA names: PEG-9 stearate to PEG-50 stearate); PEG-9 to PEG-50 palmitostearate; PEG-9 to PEG-50 behenate (as the CTFA names: PEG-9 behenate to PEG-50 behenate); polyethylene glycol 100 EO monostearate (CTFA name: PEG-100 stearate); and combinations thereof.

In a preferred embodiment, the cosmetic composition includes one or more glyceryl esters of fatty acids, for example a glyceryl stearate (glyceryl mono-, di- and/or tristearate) (CTFA name: glyceryl stearate) or glyceryl ricinoleate and combinations thereof can in particular be cited. As glyceryl esters of C8-C24 alkoxylated fatty acids, polyethoxylated glyceryl stearate (glyceryl mono-, di- and/or tristearate) such as PEG-20 glyceryl stearate and PEG-30 glyceryl stearate can for example be cited. Mixtures of these surfactants, such as for example the product containing glyceryl stearate and PEG-100 stearate, marketed under the name ARLACEL 165 by Uniqema, and the product containing glyceryl stearate (glyceryl mono- and distearate) and potassium stearate marketed under the name TEG1N by Goldschmidt (CTFA name: glyceryl stearate SE), can also be used.

Suitable alkoxylated monoacid emulsifiers include, but are not limited to: butoxynol-5 carboxylic acid, butoxynol-19 carboxylic acid, capryleth-4 carboxylic acid, capryleth-6 carboxylic Acid, capryleth-9 carboxylic acid, ceteareth-25 carboxylic acid, coceth-7 carboxylic acid, C9-11 pareth-6 carboxylic acid, C11-15 pareth-7 carboxylic acid, C12-13 pareth-5 carboxylic acid, C12-13 pareth-8 carboxylic acid, C12-13 pareth-12 carboxylic acid, C12-15 pareth-7 carboxylic acid, C12-15 pareth-8 carboxylic acid, C14-15 pareth-8 carboxylic acid, deceth-7 carboxylic acid, laureth-3 carboxylic acid, laureth-4 carboxylic Acid, laureth-5 carboxylic acid, laureth-6 carboxylic acid, laureth-8 carboxylic acid, laureth-10 carboxylic acid, laureth-11 carboxylic acid, laureth-12 carboxylic acid, laureth-13 carboxylic acid, laureth-14 carboxylic acid, laureth-17 carboxylic acid, PPG-6-laureth-6 carboxylic acid, PPG-8-steareth-7 carboxylic acid, myreth-3 carboxylic acid, myreth-5 carboxylic acid, Nonoxynol-5 carboxylic acid, nonoxynol-8 carboxylic acid, Nonoxynol-10 carboxylic acid, octeth-3 carboxylic acid, octoxynol-20 carboxylic acid, oleth-3 carboxylic acid, oleth-6 carboxylic acid, oleth-10 carboxylic acid, PPG-3-deceth-2 carboxylic acid, capryleth-2 carboxylic acid, Ceteth-13 carboxylic acid, deceth-2 carboxylic acid, hexeth-4 carboxylic acid, isosteareth-6 carboxylic acid, isosteareth-11 carboxylic acid, trudeceth-3 carboxylic acid, trideceth-6 carboxylic acid, trideceth-8 carboxylic acid, trideceth-12 carboxylic acid, trideceth-3 carboxylic acid, trideceth-4 carboxylic acid, trideceth-7 carboxylic acid, trideceth-15 carboxylic acid, trideceth-19 carboxylic acid, undeceth-5 carboxylic acid and combinations thereof.

The total amount of the one or more emulsifiers in the cosmetic composition will vary but is typically about 0.1 to about 10 wt. %, based on the total weight of the cosmetic composition. In further embodiments, the cosmetic composition includes about 0.1 to about 8 wt. %, about 0.1 to about 5 wt. %, about 0.1 to about 3 wt. %, about 0.5 to about 10 wt. %, about 0.5 to about 8 wt. %, about 0.5 to about 5 wt. %, about 0.5 to about 3 wt. %, about 1 to about 10 wt. %, about 1 to about 8 wt. %, about 1 to about 5 wt. %, about 1 to about 4 wt. %, about 2 to about 10 wt. %, about 2 to about 8 wt. %, or about 2 to about 5 wt. % of one or more emulsifiers, based on the total weight of the cosmetic composition. Preferably, the cosmetic composition includes about 0.5 to about 10 wt. %, more preferably about 1 to about 8 wt. %, and even more preferably, about 2 to about 6 wt. % of one or more emulsifiers, based on the total weight of the cosmetic composition.

In a preferred embodiment, the cosmetic composition includes at least one polymeric emulsifier and optionally one or more non-polymeric, nonionic emulsifiers. The total amount of the at least one polymeric emulsifier will vary but is typically about 0.05 to about 5 wt. %, based on the total weight of the cosmetic composition. In further embodiments, the cosmetic composition includes about 0.05 to about 3 wt. %, about 0.05 to about 2 wt. %, about 0.1 to about 5 wt. %, about 0.1 to about 3 wt. %, about 0.1 to about 2 wt. %, about 0.5 to about 5 wt. %, about 0.5 to about 3 wt. %, about 0.5 to about 2 wt. %, about 1 to about 5 wt. %, about 1 to about 3 wt. %, or about 1 to about 2 wt. % of one or more polymeric emulsifiers, based on the total weight of the cosmetic composition. Preferably, the cosmetic composition includes about 0.1 to about 5 wt. %, more preferably about 0.5 to about 3 wt. %, and even more preferably about 1 to about 2 wt. % of one or more polymeric emulsifiers.

### (d) Wax Particles

Waxes are fatty compounds having a higher melting point than oils. Thus, they are not liquids at ambient temperature (25° C.) and normal pressure (1 atm) but are solids at ambient temperature and normal pressure. Like oils, when added in an amount of at least 1% by weight into water at 25° C., waxes are not soluble in the water or only soluble to a level of 10% or less by weight, with respect to the weight of the wax added into the water.

Preferably, the wax particles have a melting point of at least 50° C. as measured by ASTM D 4591. Preferably, the wax particles have a melting point above 55° C., more preferably above 60° C., even more preferably above 65° C., and even more preferably above 70° C. as measured by ASTM D 4591. In various embodiments, the wax particles have a melting point of about 50 to about 150° C., about 55 to about 150° C., about 60 to about 150° C., about 65 to about 150° C., about 50 to about 120° C., about 55 to about 120° C., about 60 to about 120° C., about 65 to about 120° C., 50 to about 110° C., about 55 to about 150° C., about 60 to about 150° C., about 65 to about 150° C., about 50 to about 110° C., about 55 to about 110° C., about 60 to about 110° C., about 65 to about 110° C. as measured by ASTM D 4591. Preferably, the wax particles have a melting point of about 50 to about 150° C., preferably about 55 to about 130° C., more preferably about 60 to about 120° C., and even more preferably, about 65 to about 110° C. as measured by ASTM D 4591.

Nonlimiting examples of natural waxes include beeswax, carnauba wax, candelilla wax, jojoba wax, montan wax, peat wax, ouricury wax, soy wax, esparto wax, rice wax, sugar cane wax, maize wax, bayberry, rice bran wax, sunflower wax, shellac wax, castor wax, rapeseed wax, coconut wax, vegetable wax, jojoba wax, Myrica fruit wax, Myrica Cerifera (Bayberry) Fruit Wax, or combinations thereof.

Nonlimiting examples of naturally derived waxes include naturally occurring waxes which have been “substituted.” “Substituted” as used herein, means comprising at least one substituent. Nonlimiting examples of substituents include atoms, such as oxygen atoms and nitrogen atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituents may be further substituted with one of more substituents.

Waxes also include oxidized waxes, esterified waxes, etherified waxes, or combinations thereof, for example, oxidized rice bran wax or rice bran wax ethyl esters, or combinations thereof. Oxidized rice bran wax is available from Clariant (Licocare® RBW 102 VITA).

Waxes also include mono- and di-esters of ethylene glycol, diethylene glycol, triethylene glycol, for example, mono- or di-C₈-C₂₀ esters of ethylene glycol, diethylene glycol, triethylene glycol, for example, di-C₈-C₂₀ esters of ethylene glycol or di-C₁₂-C₁₈ esters of ethylene glycol. More specific nonlimiting examples include ethylene glycol distearate, ethylene glycol monostearate, ethylene glycol dipalmitate, ethylene glycol monopalmitate, ethylene glycol distearate, ethylene glycol monostearate, or combinations thereof.

In a preferred embodiment, the wax of the wax particles comprises or consists of one or more natural waxes. The one or more natural waxes are preferably selected from beeswax, carnauba wax, candelilla wax, jojoba wax, montan wax, peat wax, ouricury wax, soy wax, esparto wax, rice wax, sugar cane wax, maize wax, bayberry, rice bran wax, sunflower wax, shellac wax, castor wax, rapeseed wax, coconut wax, vegetable wax, jojoba wax, Myrica fruit wax, Myrica Cerifera (Bayberry) Fruit Wax, or combinations thereof.

The wax particles may comprise or consist of a single wax or may comprise or consist of a combination of more than one wax, provided the combination has a melting point of at least 50° C. The wax particles may include a combination of one or more waxes and one or more non-wax ingredients, provided the particles have a melting point of at least 50° C.

In a preferred embodiment, the wax particle comprise or consist of one or more waxes selected from carnauba wax, candelilla wax, bis-PEG-12 dimethicone candelillate, for instance the Siliconyl Candellila Wax sold by the company Koster Keunen, hydrogenated Jojoba wax, for instance the product sold by the company Desert Whale, hydrogenated palm oil, such as the product sold by the company SIO, rice bran wax, sumach wax, ceresin wax, laurel wax, Chinese insect wax, shellac wax, hydrogenated olive oil, such as Waxolive from the company Soliance, waxes obtained by hydrogenation of olive oil esterified with fatty alcohols comprising a C12 to C18 chain, such as those sold by the company Sophim under the trade names Phytowax® Olive 12L44, 14L48, 16L55 and 18L57, waxes obtained by hydrogenation of castor oil esterified with cetyl or behenyl alcohol, for instance those which are sold under the names Phytowax® Ricin 16 L 64 and Phytowax® Ricin 22 L 73 by the company Sophim, hydrogenated cameline wax, ouricury wax, montan wax, ozokerite waxes, for instance the Wax SP 1020 P sold by the company Strahl & Pitsch, microcrystalline waxes, for example the product sold under the trade name Microwax HW by the company Paramelt, triglycerides of lauric, palmitic, cetylic and stearic acids (INCI name: hydrogenated coco glycerides) for instance the product sold under the trade name Softisan 100 by the company Sasol, polymethylene waxes, for instance the product sold under the trade name Cirebelle 303 by the company Sasol, polyethylene waxes, for instance the products sold under the trade names Performalene 400 polyethylene, Performalene 655 polyethylene and Performalene 500-L polyethylene by the company New Phase Technologies, alcool-polyethylene waxes, for instance the product sold under the name Performacol 425 Alcohol by the company Bareco, the ethylene/acrylic acid 95/5 copolymer sold under the trade name AC 540 wax by the company Honeywell, hydroxyoctacosanyl hydroxystearate, for instance the product sold under the trade name l′Elfacos C 26 by the company Akzo, octacosanyl stearate, for instance the product sold under the name Kester Wax K 82 H by the company Koster Keunen, stearyl stearate, for instance the product sold under the name Liponate SS by the company Lipo Chemicals, pentaerythrityl distearate, for instance the product sold under the name Cutina PES by the company Cognis, the mixture of dibehenyl adipate, dioctadecyl adipate and dieicosanyl adipate (INCI name: C18-22 dialkyl adipate), the mixture of dilauryl adipate and ditetradecyl adipate (INCI name: C12-14 dialkyl adipate), the mixture of dioctadecyl sebacate, didocosyl sebacate and dieicosyl sebacate (INCI name: C18-22 dialkyl sebacate), the mixture of dioctadecyl octadecanedioate, didocosyl octanedioate and dieicosyl octanedioate (INCI name: C18-22 dialkyl octanedioate), for instance those sold by the company Cognis, pentaerythrityl tetrastearate, for instance Liponate PS-4 from the company Lipo Chemicals, tetracontanyl stearate, for instance Kester Wax K76 H from the company Koster Keunen, stearyl benzoate, for instance Finsolv 116 from the company Finetex, behenyl fumarate, for instance Marrix 222 from the company Akzo Bernel, di(trimethylol-1, 1, 1-propane) tetrastearate, for instance the product which is provided under the name Hest 2T-4S by the company Heterene, didotriacontanyl distearate, for instance Kester Wax K82D from the company Koster Keunen, polyethylene glycol montanate comprising 4 oxyethylene units (PEG-4), for instance the product sold under the name Clariant Licowax KST1, hexanediol disalycilate, for instance Betawax RX-13750 sold by the company CP Hall, dipentaerythrytyl hexastearate, for instance the product which is sold under the trade name Hest 2P-6S by the company Heterene, ditrimethylolpropane tetrabehenate, for instance the product which is sold under the trade name Hest 2T-4B by the company Heterene®, Jojoba esters, for instance the product which is sold under the trade name Floraester® HIP by the company Floratech®, mixtures of (C20-C40) linear carboxylic acid/saturated hydrocarbons (INCI name: C20-C40 acid polyethylene), for instance Performacid 350 acid from the company New Phase Technologies, synthetic wax of Fischer-Tropsch type, such as the product sold under the reference Rosswax 100 by the company Ross, stearyl alcohol, behenyl alcohol, dioctadecyl carbonate, for instance Cutina KE 3737, sucrose polybehenate, for example Crodaderm® B from the company Croda, and combinations thereof.

In a preferred embodiment, the wax particles comprise or consist one or more waxes of vegetable origin, such as carnauba wax, candelilla wax, hydrogenated jojoba wax, sumach wax, waxes obtained by hydrogenation of olive oil esterified with fatty alcohols comprising a C₁₂ to C₁₈ chain sold by the company Sophim in the Phytowax range (12L44, 14L48, 16L55 and 18L57), rice bran wax, stearyl and behenyl alcohols, laurel wax or ouricury wax.

The wax particles typically have a particle size (or number-average particle size) of about 1 to about 100 μm. In further embodiments, the particles size (or number-average particle size) is about 1 to about 50 μm, about 1 to about 25 μm, about 1 to about 20 μm, about 5 to about 100 μm, about 5 to about 50 μm, about 5 to about 25 μm, or about 5 to about 20 μm.

The particle size can be measured by various techniques, includes use of a sieve, (dynamic and static) light scattering techniques, Coulter capture techniques, measurements of sedimentation rate (related to the size via Stokes law) and microscopy. These techniques make it possible to measure a particle diameter, and for some particles, a particle size distribution. In particular, the particles sizes and the particle size distributions of the wax particles can be measured by static light scattering using a commercial particle size analyzer of MasterSizer 2000 type from Malvern. The data are processed on the basis of the Mie scattering theory. This theory, which is exact for isotropic particles, makes it possible to determine, in the case of non-spherical particles, an “effective” particle diameter. This theory is in particular described in the publication by Van de Hulst, H. C., “Light Scattering by Small Particles”, Chapters 9 and 10, Wiley, N.Y., 1957, which is incorporated herein by reference in its entirety.

The total amount of wax particles in the cosmetic composition will vary but is typically about 1 to about 15 wt. %, based on the total weight of the cosmetic composition. In further embodiments, the cosmetic composition includes about 1 to about 12 wt. %, about 1 to about 10 wt. %, about 1 to about 8 wt. %, about 2 to about 15 wt. %, about 2 to about 12 wt. %, about 2 to about 10 wt. %, about 2 to about 8 wt. %, about 3 to about 15 wt. %, about 3 to about 12 wt. %, about 3 to about 10 wt. %, about 3 to about 8 wt. %, about 5 to about 15 wt. %, about 5 to about 12 wt. %, about 5 to about 10 wt. %, or about 5 to about 8 wt. % of the wax particles, based on the total weight of the cosmetic composition. Preferably, the cosmetic composition includes about 1 to about 12 wt. %, more preferably about 2 to about 10 wt. %, and even more preferably about 3 to about 8 wt. % of the wax particles, based on the total weight of the cosmetic composition.

### (e) Soft-Focus Powder

The cosmetic composition may include one or more soft-focus powder. Soft-focus powders are materials providing a blurring effect, typically due to their light-scattering properties on the skin. Such powders typically have high diffuse reflectance, low specular reflectance, and high diffuse transmittance. Soft-focus powders give the skin a smoother appearance, for example, by reducing the difference in luminosity between the valley and the edges of wrinkles and imperfections. For purposes of the instant disclosure, the wax particles discussed above are independent and distinct from soft-focus powders.

Nonlimiting examples of soft-focus powders include powders of natural or synthetic origin such as mica, titanated mica, alumina, titanium dioxide, serecite, composite talc/titanium dioxide/alumina/silica powders, polyamide, poly(methyl (meth)acrylate), polyethylene powder, polymethylsilsesquioxane powder, sodium acrylates crosspolymer-2 and combinations thereof. Additional non-limiting examples include calcium aluminum borosilicate (LUXSIL®), PMMA (Microsphere M-100), polyethylene (POLYETHYLENE CI 2080), methyl methacrylate crosspolymer (COVABEADS LH85), nylon-12 (ORGASOL 2002), or ethylene/acrylic acid copolymer (FLOBEADS EA209).

In various embodiments, the cosmetic composition includes at least one soft-focus powder selected from silica which may or may not be coated, fumed silica, silica silylate, composite talc/titanium dioxide/alumina/silica powders, polyamide (nylon), poly(methyl (meth)acrylate), polyethylene powder, polymethylsilsesquioxane powder, dimethicone/vinyl dimethicone crosspolymer, nylon-12, cellulose, polylactic acid, boron nitride, and combinations thereof. The dimethicone/vinyl dimethicone crosspolymer can be provided as silicone dispersion (INCI: dimethicone/vinyl dimethicone crosspolymer (and) C12-14 Pareth-12). In some instances, the soft-focus powder is (or includes) sodium acrylates crosspolymer-2, which is commercially available as AQUAKEEP 10SH-NFC as sodium acrylates crosspolymer-2 (and) water (and) silica.

The total amount of soft-focus powder, if present, can vary but is typically about 0.05 to about 10 wt. %, based on the total weight of the cosmetic composition. In further embodiments, the cosmetic composition includes about 0.05 to about 8 wt. %, about 0.05 to about 5 wt. %, about 0.1 to about 10 wt. %, about 0.1 to about 8 wt. %, about 0.1 to about 5 wt. %, about 0.5 to about 10 wt. %, about 0.5 to about 8 wt. %, about 0.5 to about 5 wt. %, about 1 to about 10 wt. %, about 1 to about 8 wt. %, or about 1 to about 5 wt. % of one or more soft-focus powders, based on the total weight of the cosmetic composition. Preferably, the cosmetic composition includes about 0.01 to about 8 wt. %, more preferably about 0.05 to about 5 wt. %, and even more preferably about 0.1 to about 4 wt. % of one or more soft-focus powders, based on the total weight of the cosmetic composition.

### (f) Pigments

The cosmetic composition may include one or more pigments. Pigments include organic pigments, inorganic pigments, and combinations thereof. Various compounds serve as both pigments and as a soft-focus powder and therefore overlap exists between the description of pigments and the soft-focus powders. Nonetheless, when the cosmetic composition requires soft-focus powder and a pigment, a single compound does not serve as both the soft-focus powder and the pigment. It should be construed as one or the other.

Nonlimiting examples of organic pigments include azo pigments, polycylic pigments, anthraquinone pigments, disazo pigments, disazo condensation pigments, naphthol pigments, benzimidazolone pigments, isoindolinone pigments, isoindoline pigments, metal complex pigments, quinacridone pigments, perylene pigments, carbon black pigments, phthalocyanine pigments, perinone pigments, diketopyrrolo-pyrrole pigments, thioindigo pigments, anthropyrimidine pigments, flavanthrone pigments, anthanthrone pigments, dioxazine pigments, triarylcarbonium pigments, quinophthalone pigments, and combinations thereof.

Nonlimiting examples of inorganic pigments include titanium oxides, iron oxides, vanadium oxides, chromium oxides, manganese oxides, tungsten oxides, cadmium sulfides, zinc sulfides, cadmium mercury sulfides, iron sulfides, aluminosilicates, aluminum oxides, cerium oxides, spinel pigments, lanthanum hexaborides, and combinations and alloys thereof. In various embodiments, the cosmetic composition includes one or more inorganic pigments selected from titanium dioxide, zirconium oxide, zinc oxide, cerium oxide, magnesium oxide, barium sulfate, calcium sulfate, calcium carbonate, magnesium carbonate, talc, mica, kaolin, sericite, white mica, synthetic mica, phlogopite, lepidolite, biotite, lithia mica, silicate, silica acid anhydride, aluminum silicate, magnesium silicate, calcium silicate, barium silicate, strontium silicate, tungstic acid metal salt, hydroxyapatite, vermiculite, haidilite, bentonite, montmorillonite, hectorite, zeolite, ceramic powder, calcium phosphate, dibasic, alumina, aluminum hydroxide, boron nitride, silica, iron oxide red, iron sulfate, black iron oxide, cobalt titanate, crimson mica, or combinations thereof.

The cosmetic composition may include one or more inorganic mixed pigments. Nonlimiting examples include titanium dioxide/mica, titanium dioxide/mica/tin oxide, titanium dioxide/mica/iron oxides, titanium dioxide/mica/silicon dioxide, titanium dioxide/mica/carnine, mica/iron oxides/aluminum oxide, mica/iron oxides, titanium dioxide/mica/zinc oxide, titanium dioxide/mica/barium sulfate, mica/silicon dioxide and titanium dioxide/iron oxidesisilicon dioxide. These mixed pigments are sold under the names Timiron®, Soloron®, Colorona®, Dichrona®, Microna®, Micronaspher® and Ronaspher®. Light scattering pigments such as Ronaspher® LDP as well as nacreous pigments can also be used.

The total amount of pigments, if present, will vary but is typically about 0.01 to about 5 wt. %, based on the total weight of the cosmetic composition. In further embodiments, the cosmetic composition includes about 0.01 to about 3 wt. %, about 0.01 to about 2 wt. %, about 0.05 to about 5 wt. %, about 0.05 to about 3 wt. %, about 0.05 to about 2 wt. %, about 0.1 to about 5 wt. %, about 0.1 to about 3 wt. %, or about 0.1 to about 2 wt. % of one or more pigments, based on the total weight of the cosmetic composition. Preferably, the cosmetic composition includes about 0.01 to about 4 wt. %, preferably about 0.05 to about 3 wt. %, more preferably about 0.1 to about 2 wt. % of the one or more pigments, based on the total weight of the cosmetic composition.

As noted above, some compounds function as a pigment and as a soft-focus powder and therefore overlap exists between compounds useful as soft-focus powders and pigments. Accordingly, in a preferred embodiment, the cosmetic composition includes one or more soft-focus powders, one or more pigments, or a combination thereof. The total amount of the soft-focus powder and/or pigments will vary but is typically about 0.05 to about 10 wt. %, based on the total weight of the cosmetic composition. In further embodiments, the cosmetic composition includes about 0.05 to about 8 wt. %, about 0.05 to about 6 wt. %, about 0.1 to about 10 wt. %, about 0.1 to about 6 wt. %, about 0.1 to about 5 wt. %, about 0.5 to about 10 wt. %, about 0.5 to about 6 wt. % of soft-focus powder and/or pigments. %, based on the total weight of the cosmetic composition. Preferably, the cosmetic composition includes about 0.05 to about 10 wt. %, preferably about 0.1 to about 8 wt. %, more preferably about 0.5 to about 6 wt. % of soft-focus powder and/or pigments, based on the total weight of the cosmetic composition.

### (g) Miscellaneous Ingredients

The cosmetic composition optionally includes one or more miscellaneous ingredients. Miscellaneous ingredients are ingredients that are compatible with the cosmetic composition and do not disrupt or materially affect the basic and novel properties of the composition. Nonlimiting examples of miscellaneous ingredients include preservatives, fragrances, pH adjusters, salts, chelating agents, buffers, antioxidants, flavonoids, vitamins, botanical extracts, UV filtering agents, proteins, protein hydrolysates, and/or isolates, fillers, emollients, composition colorants, etc.

The total amount of the one or more miscellaneous ingredients will vary but is typically about 0.1 to about 10 wt. %, based on the total weight of the cosmetic composition. In further embodiments, the cosmetic composition includes about 0.1 to about 8 wt. %, about 0.1 to about 5 wt. %, about 0.5 to about 10 wt. %, about 0.5 to about 8 wt. %, about 0.5 to about 5 wt. %, about 1 to about 10 wt. %, about 1 to about 8 wt. %, about 1 to about 5 wt. %, about 2 to about 10 wt. %, about 2 to about 8 wt. %, or about 2 to about 5 wt. % of one or more miscellaneous ingredients, based on the total weight of the cosmetic composition.

pH

The pH of the cosmetic composition will vary but is typically about 4 to about 9. In various embodiments, the pH of the cosmetic composition is from about 4 to about 8, about 4 to about 7, about 5 to about 9, about 5 to about 8, about 5 to about 7, about 6 to about 9, about 6 to about 8, or about 6 to about 7. Preferably, the pH of the cosmetic composition is from about 5 to about 8, preferably from about 5 to about 7, and more preferably about 6 to about 7.

### Methods

The instant disclosure also relates to methods for treating skin, in particular, methods for improving the appearance of skin. The methods include topically applying the cosmetic composition to the skin. The cosmetic composition can be topically applied directly to the skin using one's hand, a cloth, or an applicator tool, which may optionally be integrated with packing for the cosmetic composition. The skin may be washed or rinsed prior to application. Preferably, the cosmetic composition is applied to the skin of the face, including skin around the eyes. The methods improve the appearance of the skin, eliminate or reduce the appearance fine lines and/or discoloration, remove excess oil from the skin, improve skin texture and tone, hydrate the skin, obscure and/or blur imperfections of the skin, and smooth the skin.

### PREFERRED EMBODIMENTS

In a preferred embodiment, the cosmetic composition comprises, consists essentially of, or consists of:
(a) about 65 to about 90 wt. %, preferably about 70 to about 85 wt. %, more preferably about 75 to about 85 wt. % of an aqueous carrier phase, the aqueous carrier phase comprising:
   (i) about 0.1 to about 12 wt. %, preferably about 0.5 to about 8 wt. %, more preferably about 1 to about 5 wt. % of one or more hydrophilic film forming polymers, wherein the one or more hydrophilic film forming polymers are preferably selected from polyalkyloxazolines, vinyl acetate homopolymers, vinyl acetate copolymers, acrylic ester homopolymers and copolymers, copolymers of acrylonitrile and of a nonionic monomer, styrene homopolymers, styrene copolymers, copolymers of styrene and of butadiene, or copolymers of styrene, of butadiene and of vinylpyridine, polyamides, vinyllactam homopolymers, vinyllactam copolymers, vinylpyrrolidone/methacrylamide/vinylimidazole terpolymers, polyvinylpyrrolidone (PVP), vinylpyrrolidone copolymers, carboxylic acid or carboxylate-based homo- and copolymers, polysaccharides or combinations thereof, wherein preferably, at least one of the one or more hydrophilic film forming polymers is selected from polyvinylpyrrolidone (PVP), polyvinylpyrrolidone (PVP)/vinyl acetate copolymer (PVP/VA copolymer), polyvinylpyrrolidone (PVP)/eicosene copolymer, PVP/hexadecene copolymer, VP/methacrylamide/vinyl Imidazole copolymer, or combinations thereof, wherein even more preferably, at least one of the one or more hydrophilic film forming polymers is selected from vinylpyrrolidone/methacrylamide/vinyl imidazole; and
   (ii) about 1 to about 25 wt. %, preferably about 2 to about 20 wt. %, more preferably about 5 to about 15 wt. % of one or more water soluble solvents, wherein preferably, the one or more water soluble solvents are selected from glycerin, C2-C6 mono-alcohols, polyols, glycols, glycol ethers, or combinations thereof, wherein even more preferably, the one or more water soluble solvents are selected from C2-C6 mono-alcohols, glycerin, ethylene glycol, propylene glycol, butylene glycol, hexylene glycol, caprylyl glycol, dipropylene glycol, wherein more preferably, at least one, at least two or all of the one or more water soluble solvents are selected from glycerin, ethanol, isopropanol, glycerin, ethylene glycol, propylene glycol, butylene glycol, hexylene glycol, caprylyl glycol, dipropylene glycol; and
   (iii) about 40 to about 85 wt. %, preferably about 50 to about 80 wt. %, and more preferably about 50 to about 75 wt. % of water;
(b) about 3 to about 25 wt. %, preferably about 5 to about 20 wt. %, and more preferably about 5 to about 15 wt. % of an oil phase dispersed throughout the aqueous carrier phase, the oil phase comprising:
   (i) about 0.1 to about 10 wt. %, preferably about 0.5 to about 8 wt. %, and more preferably about 1 to about 5 wt. % of one or more hydrophobic film forming polymers, wherein preferably, the one or more hydrophobic film forming polymers are selected from trimethylsiloxysilicates, phenylalkylsiloxysilicates, phenylpropyldimethylsiloxysilicate; silicone acrylate polymers, acrylate/dimethicone copolymers acrylate/polytrimethylsiloxymethacrylate copolymers, acrylate/polytrimethylsiloxymethacrylate copolymers, acrylate/polytrimethylsiloxymethacrylate copolymers, polyalkylsilsesquioxanes, polymethylsilsesquioxane, trialkylsiloxysilylcarbamoyl pullulans having an alkyl group comprising from 1 to 6 carbon atoms, dimethicone copolymers, dimethicone crosspolymer, copolymers of vinylpyrrolidone (VP) and alkene comprising 2 to 20 carbon atoms, copolymers of a vinyl ester, hydrogenated or non-hydrogenated polyolefins, or combinations thereof, wherein preferably, the one or more hydrophobic film forming polymers are selected from trimethylsiloxysilicate, acrylates/isobornyl acrylate copolymer, acrylates/stearyl methacrylate copolymer, acrylic acid/isobutyl acrylate/isobornyl acrylate copolymer, acrylates/stearyl methacrylate copolymer, acrylate/polytrimethyl siloxymethacrylate copolymer, C30-45 alkyldimethylsilylpolypropylsilsequixane, trimethylsilsesquixane, polypropylsilsesquixane, acrylates/dimethicone copolymer, octylacrylamide/acrylates/butylaminoethyl methacrylate copolymer, VA/butyl maleate/isobornyl acrylate copolymer, acrylates/t-butylacrylamide copolymer, polyvinylpyrrolidone/vinyl acetate copolymer, triacontanyl PVP copolymer, acrylates/dimethylaminoethyl methacrylate copolymer, norbornene/tris(trimethylsiloxy) silylnorbornenecopolymer, styrene/butadiene copolymer, trimethylsiloxysilylcarbamoyl pullulan, wherein even more preferably the one or more hydrophobic film forming polymers are selected from trimethylsiloxysilylcarbamoyl pullulan, acrylates/isobornyl acrylate copolymer, acrylates/stearyl methacrylate copolymer, dimethicone crosspolymer, or combinations thereof;
   (ii) about 1 to about 20 wt. %, preferably about 2 to about 15 wt. %, and even more preferably about 3 to about 12 wt. % of one or more volatile hydrocarbon oils selected from polyisobutene, isoparaffin, isohexadecane, isododecane, isodecane, isohexane, undecane, tridecane, dodecane, C13-C14 isoalkane, or combinations thereof;
(c) about 0.1 to about 10 wt. %, preferably about 0.5 to about 8 wt. %, even more preferably about 1 to about 5 wt. % of one or more emulsifiers, wherein preferably at least one of the one or more emulsifiers is a polymeric emulsifiers, preferably selected from poloxamers (ethoxylated sorbitan), polyamide, alkyl and polyalkyl ethers of poly(ethylene oxide), taurate copolymers, or combinations thereof;
(d) about 0.1 to about 12, preferably about 1 to about 8, more preferably about 2 to about 8 wt. % of discrete wax particles dispersed throughout the aqueous carrier phase, wherein the discrete wax particles have an average particle size of about 1 μm to about 100 μm, preferably about 1 μm to about 50 μm, more preferably about 1 μm to about 20 μm, and a melting point greater than 50° C., preferably greater than 55° C., and more preferably greater than 60° C., wherein preferably, the wax particles comprise or consist of a natural wax, preferably a natural wax selected from beeswax, carnauba wax, candelilla wax, jojoba wax, montan wax, peat wax, ouricury wax, soy wax, esparto wax, rice wax, sugar cane wax, maize wax, bayberry, rice bran wax, sunflower wax, shellac wax, castor wax, rapeseed wax, coconut wax, vegetable wax, jojoba wax, Myrica fruit wax, Myrica Cerifera (Bayberry) Fruit Wax, or combinations thereof, wherein even more preferably, the discrete wax particle comprise or consist of Carnauba wax; and
(e) optionally, about 0.05 to about 8 wt. %, preferably about 0.1 to about 5 wt. %, and more preferably about 1 to about 5 wt. % of one or more soft-focus powder, preferably wherein the one or more soft focus powders are selected from silica, fumed silica, silica silylate, composite talc/titanium dioxide/alumina/silica powders, polyamide (nylon), poly(methyl (meth)acrylate), polyethylene powder, polymethylsilsesquioxane powder, dimethicone/vinyl dimethicone crosspolymer, nylon-12, cellulose, polylactic acid, boron nitride, and combinations thereof, wherein more preferably, the one or more soft-focus powders are selected from silica, silica silylate, cellulose, or combinations thereof;
(f) optionally, about 0.01 to about 5 wt. %, preferably about 0.05 to about 4 wt. %, more preferably about 0.1 to about 3 wt. % of one or more pigments, preferably, one or more inorganic pigments, wherein preferably, the one or more inorganic pigments are selected from mica, titanium dioxide, zirconium oxide, zinc oxide, cerium oxide, magnesium oxide, barium sulfate, calcium sulfate, calcium carbonate, magnesium carbonate, talc, mica, kaolin, sericite, white mica, synthetic mica, phlogopite, lepidolite, biotite, lithia mica, silicate, silica acid anhydride, aluminum silicate, magnesium silicate, calcium silicate, barium silicate, strontium silicate, tungstic acid metal salt, hydroxyapatite, vermiculite, haidilite, bentonite, montmorillonite, hectorite, zeolite, ceramic powder, calcium phosphate, dibasic, alumina, aluminum hydroxide, boron nitride, silica, iron oxide, iron sulfate, black iron oxide, cobalt titanate, crimson mica, or combinations thereof;
(g) optionally, about 0.01 to about 10 wt. %, preferably about 0.1 to about 8 wt. %, more preferably about 1 to about 6 wt. % of one or more miscellaneous ingredients, wherein preferably, the one or more miscellaneous ingredients are selected from preservatives, fragrances, essential oils, chelating agents, antioxidants, flavonoids, vitamins, botanical extracts, UV filtering agents, proteins, protein hydrolysates, protein isolates, fillers, composition colorants, or combinations thereof;
   wherein the cosmetic composition provides an immediate skin tightening effect upon application to the skin, and
   all weight percentages are based on a total weight of the composition.

In another preferred embodiment, the cosmetic composition comprises, consists essentially of, or consists of:
(a) about 65 to about 90 wt. %, preferably about 70 to about 85 wt. %, more preferably about 75 to about 85 wt. % of an aqueous carrier phase, the aqueous carrier phase comprising:
   (i) about 0.1 to about 12 wt. %, preferably about 0.5 to about 8 wt. %, more preferably about 1 to about 5 wt. % of one or more hydrophilic film forming polymers selected from polyalkyloxazolines, vinyl acetate homopolymers, vinyl acetate copolymers, acrylic ester homopolymers and copolymers, copolymers of acrylonitrile and of a nonionic monomer, styrene homopolymers, styrene copolymers, copolymers of styrene and of butadiene, or copolymers of styrene, of butadiene and of vinylpyridine, polyamides, vinyllactam homopolymers, vinyllactam copolymers, vinylpyrrolidone/methacrylamide/vinylimidazole terpolymers, polyvinylpyrrolidone (PVP), vinylpyrrolidone copolymers, carboxylic acid or carboxylate-based homo- and copolymers, or combinations thereof, wherein preferably, at least one of the one or more hydrophilic film forming polymers is selected from polyvinylpyrrolidone (PVP), polyvinylpyrrolidone (PVP)/vinyl acetate copolymer (PVP/VA copolymer), polyvinylpyrrolidone (PVP)/eicosene copolymer, PVP/hexadecene copolymer, VP/methacrylamide/vinyl Imidazole copolymer, or combinations thereof, wherein even more preferably, at least one of the one or more hydrophilic film forming polymers is selected from vinylpyrrolidone/methacrylamide/vinyl imidazole; and
   (ii) about 1 to about 25 wt. %, preferably about 2 to about 20 wt. %, more preferably about 5 to about 15 wt. % of one or more water soluble solvents selected from glycerin, C2-C6 mono-alcohols, ethylene glycol, propylene glycol, butylene glycol, hexylene glycol, caprylyl glycol, dipropylene glycol, wherein more preferably, at least one, at least two or all of the one or more water soluble solvents are selected from glycerin, ethanol, isopropanol, glycerin, ethylene glycol, propylene glycol, butylene glycol, hexylene glycol, caprylyl glycol, dipropylene glycol; and
   (iii) about 40 to about 85 wt. %, preferably about 50 to about 80 wt. %, and more preferably about 50 to about 75 wt. % of water;
(b) about 3 to about 25 wt. %, preferably about 5 to about 20 wt. %, and more preferably about 5 to about 15 wt. % of an oil phase dispersed throughout the aqueous carrier phase, the oil phase comprising:
   (i) about 0.1 to about 10 wt. %, preferably about 0.5 to about 8 wt. %, and more preferably about 1 to about 5 wt. % of one or more hydrophobic film forming polymers selected from trimethylsiloxysilicates, phenylalkylsiloxysilicates, phenylpropyldimethylsiloxysilicate; silicone acrylate polymers, acrylate/dimethicone copolymers acrylate/polytrimethylsiloxymethacrylate copolymers, acrylate/polytrimethylsiloxymethacrylate copolymers, acrylate/polytrimethylsiloxymethacrylate copolymers, polyalkylsilsesquioxanes, polymethylsilsesquioxane, trialkylsiloxysilylcarbamoyl pullulans having an alkyl group comprising from 1 to 6 carbon atoms, dimethicone copolymers, dimethicone crosspolymer, copolymers of vinylpyrrolidone (VP) and alkene comprising 2 to 20 carbon atoms, copolymers of a vinyl ester, hydrogenated or non-hydrogenated polyolefins, or combinations thereof, wherein preferably, the one or more hydrophobic film forming polymers are selected from trimethylsiloxysilicate, acrylates/isobornyl acrylate copolymer, acrylates/stearyl methacrylate copolymer, acrylic acid/isobutyl acrylate/isobornyl acrylate copolymer, acrylates/stearyl methacrylate copolymer, acrylate/polytrimethyl siloxymethacrylate copolymer, C30-45 alkyldimethylsilylpolypropylsilsequixane, trimethylsilsesquixane, polypropylsilsesquixane, acrylates/dimethicone copolymer, octylacrylamide/acrylates/butylaminoethyl methacrylate copolymer, VA/butyl maleate/isobornyl acrylate copolymer, acrylates/t-butylacrylamide copolymer, polyvinylpyrrolidone/vinyl acetate copolymer, triacontanyl PVP copolymer, acrylates/dimethylaminoethyl methacrylate copolymer, norbornene/tris(trimethylsiloxy) silylnorbornenecopolymer, styrene/butadiene copolymer, trimethylsiloxysilylcarbamoyl pullulan, wherein even more preferably the one or more hydrophobic film forming polymers are selected from trimethylsiloxysilylcarbamoyl pullulan, acrylates/isobornyl acrylate copolymer, acrylates/stearyl methacrylate copolymer, dimethicone crosspolymer, or combinations thereof;
   (ii) about 1 to about 20 wt. %, preferably about 2 to about 15 wt. %, and even more preferably about 3 to about 12 wt. % of one or more volatile hydrocarbon oils selected from polyisobutene, isoparaffin, isohexadecane, isododecane, isodecane, isohexane, undecane, tridecane, dodecane, C13-C14 isoalkane, or combinations thereof;
(c) about 0.1 to about 10 wt. %, preferably about 0.5 to about 8 wt. %, even more preferably about 1 to about 5 wt. % of one or more polymeric emulsifiers and one or more nonionic emulsifiers, wherein preferably the one or more polymeric emulsifiers are selected from polyamide, poloxamers (ethoxylated sorbitan), alkyl and polyalkyl ethers of poly(ethylene oxide), taurate copolymers, or combinations thereof, preferably wherein the one or more polymeric emulsifiers includes acrylamide/sodium acryloyldimethyltaurate copolymer;
(d) about 0.1 to about 12, preferably about 1 to about 8, more preferably about 2 to about 8 wt. % of discrete wax particles dispersed throughout the aqueous carrier phase, wherein the discrete wax particles have an average particle size of about 1 μm to about 100 μm, preferably about 1 μm to about 50 μm, more preferably about 1 μm to about 20 μm, and a melting point greater than 50° C., preferably greater than 55° C., and more preferably greater than 60° C., wherein preferably, the wax particles comprise or consist of a natural wax, preferably a natural wax selected from beeswax, carnauba wax, candelilla wax, jojoba wax, montan wax, peat wax, ouricury wax, soy wax, esparto wax, rice wax, sugar cane wax, maize wax, bayberry, rice bran wax, sunflower wax, shellac wax, castor wax, rapeseed wax, coconut wax, vegetable wax, jojoba wax, Myrica fruit wax, Myrica Cerifera (Bayberry) Fruit Wax, or combinations thereof, wherein even more preferably, the discrete wax particle comprise or consist of Carnauba wax; and
(e) optionally, about 0.05 to about 8 wt. %, preferably about 0.1 to about 5 wt. %, and more preferably about 1 to about 5 wt. % of one or more soft-focus powder, preferably wherein the one or more soft focus powders are selected from silica, fumed silica, silica silylate, composite talc/titanium dioxide/alumina/silica powders, polyamide (nylon), poly(methyl(meth)acrylate), polyethylene powder, polymethylsilsesquioxane powder, dimethicone/vinyl dimethicone crosspolymer, nylon-12, cellulose, polylactic acid, boron nitride, and combinations thereof, wherein more preferably, the one or more soft-focus powders are selected from silica, silica silylate, cellulose, or combinations thereof;
(f) optionally, about 0.01 to about 5 wt. %, preferably about 0.05 to about 4 wt. %, more preferably about 0.1 to about 3 wt. % of one or more pigments, preferably, one or more inorganic pigments, wherein preferably, the one or more inorganic pigments are selected from mica, titanium dioxide, zirconium oxide, zinc oxide, cerium oxide, magnesium oxide, barium sulfate, calcium sulfate, calcium carbonate, magnesium carbonate, talc, mica, kaolin, sericite, white mica, synthetic mica, phlogopite, lepidolite, biotite, lithia mica, silicate, silica acid anhydride, aluminum silicate, magnesium silicate, calcium silicate, barium silicate, strontium silicate, tungstic acid metal salt, hydroxyapatite, vermiculite, haidilite, bentonite, montmorillonite, hectorite, zeolite, ceramic powder, calcium phosphate, dibasic, alumina, aluminum hydroxide, boron nitride, silica, iron oxide, iron sulfate, black iron oxide, cobalt titanate, crimson mica, or combinations thereof;
(g) optionally, about 0.01 to about 10 wt. %, preferably about 0.1 to about 8 wt. %, more preferably about 1 to about 6 wt. % of one or more miscellaneous ingredients, wherein preferably, the one or more miscellaneous ingredients are selected from preservatives, fragrances, essential oils, chelating agents, antioxidants, flavonoids, vitamins, botanical extracts, UV filtering agents, proteins, protein hydrolysates, protein isolates, fillers, composition colorants, or combinations thereof;
   wherein the cosmetic composition provides an immediate skin tightening effect upon application to the skin, and
   all weight percentages are based on a total weight of the composition.

In yet a further preferred embodiment, the cosmetic composition comprises, consists essentially of, or consists of:
(a) about 65 to about 90 wt. %, preferably about 70 to about 85 wt. %, more preferably about 75 to about 85 wt. % of an aqueous carrier phase, the aqueous carrier phase comprising:
   (i) about 0.1 to about 12 wt. %, preferably about 0.5 to about 8 wt. %, more preferably about 1 to about 5 wt. % of vinylpyrrolidone/methacrylamide/vinyl imidazole;
   (ii) about 1 to about 25 wt. %, preferably about 2 to about 20 wt. %, more preferably about 5 to about 15 wt. % of one or more water soluble solvents selected from glycerin, C2-C6 mono-alcohols, ethylene glycol, propylene glycol, butylene glycol, hexylene glycol, caprylyl glycol, dipropylene glycol; and
   (iii) about 40 to about 85 wt. %, preferably about 50 to about 80 wt. %, and more preferably about 50 to about 75 wt. % of water;
(b) about 3 to about 25 wt. %, preferably about 5 to about 20 wt. %, and more preferably about 5 to about 15 wt. % of an oil phase dispersed throughout the aqueous carrier phase, the oil phase comprising:
   (i) about 0.1 to about 10 wt. %, preferably about 0.5 to about 8 wt. %, and more preferably about 1 to about 5 wt. % of one or more hydrophobic film forming polymers selected from dimethicone crosspolymer, acrylates/isobornyl acrylate copolymer, acrylates/stearyl methacrylate copolymer, dimethicone crosspolymer, or combinations thereof;
   (ii) about 1 to about 20 wt. %, preferably about 2 to about 15 wt. %, and even more preferably about 3 to about 12 wt. % of one or more volatile hydrocarbon oils selected from polyisobutene, isoparaffin, isohexadecane, isododecane, isodecane, isohexane, undecane, tridecane, dodecane, C13-C14 isoalkane, or combinations thereof, preferably isododecane, isohexadecane, C13-C14 isoalkane, or combinations thereof;
(c) about 0.1 to about 10 wt. %, preferably about 0.5 to about 8 wt. %, even more preferably about 1 to about 5 wt. % of one or more polymeric emulsifiers and one or more nonionic emulsifiers, wherein preferably the one or more polymeric emulsifiers are selected from polyamide, poloxamers (ethoxylated sorbitan), alkyl and polyalkyl ethers of poly(ethylene oxide), taurate copolymers, or combinations thereof, preferably wherein the one or more polymeric emulsifiers includes acrylamide/sodium acryloyldimethyltaurate copolymer, a polysorbate, or a combination thereof;
(d) about 0.1 to about 12, preferably about 1 to about 8, more preferably about 2 to about 8 wt. % of discrete wax particles dispersed throughout the aqueous carrier phase, wherein the discrete wax particles have an average particle size of about 1 μm to about 100 μm, preferably about 1 μm to about 50 μm, more preferably about 1 μm to about 20 μm, and a melting point greater than 50° C., preferably greater than 55° C., and more preferably greater than 60° C., wherein preferably, the wax particles comprise or consist of a natural wax, preferably a natural wax selected from beeswax, carnauba wax, candelilla wax, jojoba wax, montan wax, peat wax, ouricury wax, soy wax, esparto wax, rice wax, sugar cane wax, maize wax, bayberry, rice bran wax, sunflower wax, shellac wax, castor wax, rapeseed wax, coconut wax, vegetable wax, jojoba wax, Myrica fruit wax, Myrica Cerifera (Bayberry) Fruit Wax, or combinations thereof, wherein even more preferably, the discrete wax particle comprise or consist of Carnauba wax; and
(e) optionally, about 0.05 to about 8 wt. %, preferably about 0.1 to about 5 wt. %, and more preferably about 1 to about 5 wt. % of one or more soft-focus powder, preferably wherein the one or more soft focus powders are selected from silica, fumed silica, silica silylate, composite talc/titanium dioxide/alumina/silica powders, polyamide (nylon), poly(methyl (meth)acrylate), polyethylene powder, polymethylsilsesquioxane powder, dimethicone/vinyl dimethicone crosspolymer, nylon-12, cellulose, polylactic acid, boron nitride, and combinations thereof, wherein more preferably, the one or more soft-focus powders are selected from silica, silica silylate, cellulose, or combinations thereof;
(f) optionally, about 0.01 to about 5 wt. %, preferably about 0.05 to about 4 wt. %, more preferably about 0.1 to about 3 wt. % of one or more pigments, preferably, one or more inorganic pigments, wherein preferably, the one or more inorganic pigments are selected from mica, titanium dioxide, zirconium oxide, zinc oxide, cerium oxide, magnesium oxide, barium sulfate, calcium sulfate, calcium carbonate, magnesium carbonate, talc, mica, kaolin, sericite, white mica, synthetic mica, phlogopite, lepidolite, biotite, lithia mica, silicate, silica acid anhydride, aluminum silicate, magnesium silicate, calcium silicate, barium silicate, strontium silicate, tungstic acid metal salt, hydroxyapatite, vermiculite, haidilite, bentonite, montmorillonite, hectorite, zeolite, ceramic powder, calcium phosphate, dibasic, alumina, aluminum hydroxide, boron nitride, silica, iron oxide, iron sulfate, black iron oxide, cobalt titanate, crimson mica, or combinations thereof;
(g) optionally, about 0.01 to about 10 wt. %, preferably about 0.1 to about 8 wt. %, more preferably about 1 to about 6 wt. % of one or more miscellaneous ingredients, wherein preferably, the one or more miscellaneous ingredients are selected from preservatives, fragrances, essential oils, chelating agents, antioxidants, flavonoids, vitamins, botanical extracts, UV filtering agents, proteins, protein hydrolysates, protein isolates, fillers, composition colorants, or combinations thereof;
   wherein the cosmetic composition provides an immediate skin tightening effect upon application to the skin, and
   all weight percentages are based on a total weight of the composition.

### EXAMPLES

Various changes can be made in the above-compositions and methods without departing from the scope of the invention. Accordingly, it is intended that the entire disclosure in the above description and in the examples given below, is illustrative and not limiting.

### Example 1

### Inventive Compositions

| | | | | A | B | C | D | E | F |
|---|---|---|---|---|---|---|---|---|---|
| (a) | (i) | Film | VP/METHACRYLAMIDE/ VINYL | 3 | 3 | 3 | 2.4 | 1.2 | 1.2 |
| Aqueous | | Former | IMIDAZOLE COPOLYMER | | | | | | |
| Phase | (ii) | Water | ALCOHOL DENAT. | 11.9 | 22.2 | 12.2 | 12.2 | 7.2 | |
| | | Soluble | BUTYLENE GLYCOL, | 0.2 | 2.2 | 2.5 | 0.3 | 2.5 | 2.9 |
| | | Solvent | CAPRYLYL GLYCOL, AND/OR | | | | | | |
| | | | PROPANEDIOL | | | | | | |
| | | | GLYCERIN | | | | 1 | | |
| | (iii) | Water | WATER | 67.9 | 52.1 | 61.2 | 63.4 | 72.8 | 69 |
| Total Aqueous Carrier Phase | | | | 83 | 80 | 79 | 79 | 84 | 74 |
| (b) | (i) | Film | ACRYLATES/ISOBORNYL | | | | | | 0.5 |
| Oil | | Former | ACRYLATE COPOLYMER | | | | | | |
| Phase | | | ACRYLATES/STEARYL | | | | | 0.5 | |
| | | | METHACRYLATE COPOLYMER | | | | | | |
| | | | TRIMETHYLSILOXY- | 2.4 | 2.4 | 2.4 | 2.4 | 0.7 | 0.7 |
| | | | SILYLCARBAMOYL PULLULAN | | | | | | |
| | | | DIMETHICONE | | 0.2 | 0.2 | 0.2 | 0.2 | 0.4 |
| | | | CROSSPOLYMER | | | | | | |
| | (ii) | Volatile | ISODODECANE | 5.6 | 6.4 | 6.4 | 6.4 | 2.9 | 8.8 |
| | | Oil | ISOHEXADECANE | 0.6 | 0.6 | 0.6 | 0.6 | 0.6 | |
| | | | C13-14 ISOALKANE | | | | | | 0.6 |
| Weight Ratio of (a)(i):(b)(i) | | | | 1.3:1 | 1.3:1 | 1.3:1 | 1:1 | 1:1.2 | 1:1.3 |
| Weight Ratio of (b)(i):(b)(ii) | | | | 1:2.6 | 1:2.7 | 1:2.7 | 1:2.7 | 1:2.5 | 1:5.9 |
| Total Oil Phase | | | | 8.6 | 9.6 | 9.6 | 9.6 | 4.9 | 11 |
| (c) | | Emulsifiers | ACRYLAMIDE/SODIUM | 1.2 | 1.2 | 1.2 | 1.2 | 1.2 | |
| | | | ACRYLOYLDIMETHYL- | | | | | | |
| | | | TAURATE COPOLYMER | | | | | | |
| | | | POLYACRYLAMIDE | | | | | | 1.2 |
| | | | POLYSORBATE 80, PEG-30 | 1.6 | 1.9 | 1.9 | 1.9 | 1.9 | 1.8 |
| | | | GLYCERYL STEARATE, | | | | | | |
| | | | SORBITAN OLEATE, AND /OR | | | | | | |
| | | | LAURETH-7 | | | | | | |
| (d) | | Wax | CARNAUBA WAX PARTICLES | 5 | 6 | 6 | 6 | 6 | 6 |
| | | Particles | | | | | | | |
| (e) | | Soft-Focus | SILICA | 0.5 | 0.7 | 0.7 | 0.7 | 0.7 | 2.5 |
| | | Powder | SILICA SILYLATE | | | | | 0.1 | |
| | | | CELLULOSE | | 1 | 1 | 1 | 1 | 2.2 |
| (f) | | Pigments | MICA, TITANIUM DIOXIDE, | | | | | | 0.7 |
| | | | AND/OR IRON OXIDE | | | | | | |
| | | | BORON NITRIDE | | | | | | 0.2 |
| | | | BARIUM SULFATE | | | | | | 0.3 |

| | | | | | | | |
|---|---|---|---|---|---|---|---|
| (g) | Miscellaneous* | ≤5 | ≤5 | ≤5 | ≤5 | ≤5 | ≤5 |

*Miscellaneous ingredients such as preservatives, fragrances, pH adjusters, salts, chelating agents, buffers, antioxidants, flavonoids, vitamins, botanical extracts, UV filtering agents, proteins, protein hydrolysates, and/or isolates, fillers, emollients, composition colorants, or combinations thereof.

### Example 2

### Performance Evaluation

Inventive Compositions C, E, and F were evaluated and compared with two commercial benchmark products, which are advertised as skin smoothing compositions that blur lines, wrinkles, and pores, and smooth uneven skin textures. Equal amounts of each composition (10 mg/cm²) were applied to a stretchable substrate (die-cut 2″×1″ Supplale® substrate strips) and allowed to dry overnight. The cosmetic compositions were applied at a wet thickness of about 2 mm. The samples were subsequently evaluated for internal stress and cracking. Internal stress was determined by comparing the two-dimensional area of the coated Supplale® substrate strips to a blank Supplale® substrate strip. The internal stress is represented as % reduction of the two-dimensional area. Cracking was assessed after stretching the coated strips. The coated strips were stretched to 25% extension from original length in the direction of stretch. The degree of cracking was visually evaluated and grated on a scale of 0 to 5, where 0 represents no cracking and 5 represents more cracking. The results are presented in the table below.

| | Internal | Cracking |
|---|---|---|
| Composition | Stress (%) | (score) |
| Inventive C | 37.3 | 5 |
| Inventive E | 24.3 | 2 |
| Inventive F | 37.9 | 5 |
| Commercial Benchmark 1 | 0 | 0 |
| Commercial Benchmark 2 | 0 | 4 |

The data show that, unlike both commercial benchmark products, the inventive compositions provide a significant “tightening” effect. This is illustrated by the data for internal stress. Commercial benchmark 1 did not exhibit cracking upon stretching. Commercial benchmark 2, however, exhibited cracking even though commercial benchmark 2 did not provide any tightening. Tightening is associated with cracking, i.e., the more a composition tightens and contracts, the more likely it will suffer cracking when stretched. This is illustrated by the data for the inventive compositions C, E, and F. Inventive compositions C and F with the highest values for internal stress (illustrating the most tightening) exhibited the most cracking. Inventive composition D having a lower percentage of internal stress exhibited less cracking. Ideally, the cosmetic composition should provide significant tightening without an unacceptable degree of cracking when stretched. This is the case for inventive compositions C, E, and F. The inventive compositions provide significant tightening. They show cracking when stretched but the degree of cracking is acceptable, as illustrated by commercial benchmark 2, which exhibited cracking even though it failed to provide a tightening effect.

### Example 3

### Contribution of Discrete Wax Particles

| | | | | C-1 |
|---|---|---|---|---|
| (a) | (i) | Film | VP/METHACRYLAMIDE/ | 1.2 |
| Aqueous | | Former | VINYL IMIDAZOLE | |
| Phase | | | COPOLYMER | |
| | (ii) | Water | BUTYLENE GLYCOL, | 2.9 |
| | | Soluble | CAPRYLYL GLYCOL, | |
| | | Solvent | AND/OR PROPANEDIOL | |
| | (iii) | Water | WATER | 74 |
| Total Aqueous Carrier Phase | | | | 78 |
| (b) | (i) | Film | ACRYLATES/ISOBORNYL | 0.5 |
| Oil | | Former | ACRYLATE COPOLYMER | |
| Phase | | | TRIMETHYLSILOXY- | 0.7 |
| | | | SILYLCARBAMOYL | |
| | | | PULLULAN | |
| | | | DIMETHICONE | 0.2 |
| | | | CROSSPOLYMER | |
| | (ii) | Volatile | ISODODECANE | 1.3 |
| | | Oil | UNDECANE | 7.1 |
| | | | C13-14 ISOALKANE | 0.7 |
| | | | TRIDECANE | 3.5 |

| Weight Ratio of (a)(i):(b)(i) | | | 1:1.3 |
|---|---|---|---|
| Weight Ratio of (b)(i):(b)(ii) | | | 1:9 |
| Total Oil Phase | | | 14 |
| (c) | Emulsifiers | POLYACRYLAMIDE | 1.2 |
| | | LAURETH-7 | 0.2 |
| (d) | Wax | CARNAUBA WAX | |
| | Particles | PARTICLES | |
| (e) | Soft-Focus | SILICA | 1.5 |
| | Powder | CELLULOSE | 2 |
| (f) | Pigments | MICA, TITANIUM DIOXIDE, | 0.7 |
| | | AND/OR IRON OXIDE | |
| | | BORON NITRIDE | 0.2 |
| | | BARIUM SULFATE | 0.3 |

| | | |
|---|---|---|
| (g) | Miscellaneous* | ≤5 |

*Miscellaneous ingredients such as preservatives, fragrances, pH adjusters, salts, chelating agents, buffers, antioxidants, flavonoids, vitamins, botanical extracts, UV filtering agents, proteins, protein hydrolysates, and/or isolates, fillers, emollients, composition colorants, or combinations thereof.

Comparative Composition C-1 is similar to Inventive Composition F of Example 1 but does not include the discrete wax particles. Internal stress and cracking were evaluated as described in Example 2. The results are shown in the table below.

| | Internal | Cracking |
|---|---|---|
| Composition | Stress (%) | (score) |
| Inventive F | 37.9 | 5 |
| Comparative Composition C-1 | 1.1 | 1 |

The data show that Comparative Composition C-1, lacking the discrete wax particles, failed to provide tightening/contraction.

The foregoing disclosure illustrates and describes embodiments of the invention. The disclosure shows and describes only the preferred embodiments but it is understood that the invention is useable in various other combinations, modifications, and environments and is capable of changes or modifications within the scope of the inventive concepts as expressed herein, commensurate with the above teachings and/or the skill or knowledge of the relevant art. The embodiments described throughout the disclosure are intended to explain best modes known by applicant to enable others skilled in the art to utilize the invention but is not intended to limit the invention.

As used herein, the terms “comprising,” “having,” and “including” (or “comprise,” “have,” and “include”) are used in their open, non-limiting sense. The phrase “consisting essentially of” limits the scope of a claim to the specified materials or steps and those that do not materially affect the basic and novel characteristics of the claimed invention.

The terms “a,” “an,” and “the” are understood to encompass the plural as well as the singular.

Thus, the term “a mixture thereof” or “a combination thereof” also relates to “mixtures thereof” or “combinations thereof.” Throughout the disclosure, the term “a combination thereof” may be used following a list of elements as shown in the following example where letters A-F represent the elements: “one or more elements selected from the group consisting of A, B, C, D, E, F, and a combination thereof.” The term, “a combination thereof” does not require that the combination include all of A, B, C, D, E, and F (although all of A, B, C, D, E, and F may be included). Rather, it indicates that a combination of any two or more of A, B, C, D, E, and F can be included. In other words, it is equivalent to the phrase “one or more elements selected from the group consisting of A, B, C, D, E, F, and a combination of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, and a combination thereof,” it indicates that that one or more of A, B, C, D, and F may be included, one or more of a salt of A, a salt of B, a salt of C, a salt of D, a salt of E, and a salt of F may be include, or a mixture of any two of A, B, C, D, E, F, a salt of A, a salt of B, a salt of C, a salt of D, a salt of E, and a salt of F may be included.

The term “polymer,” as used throughout the disclosure is a generic term that includes both homopolymers and copolymers.

The language throughout the disclosure referring to the cosmetic composition imparting or providing “an immediate skin tightening effect” or “an instantaneous skin tightening effect” means that upon application of a layer of the cosmetic composition onto skin or onto a substrate (applied with a wet thickness of about 2 mm) under ambient conditions (about 25° C.), the cosmetic composition will tighten or contract as it dries on the skin or substrate. The tightening or contraction is observed (seen with the naked eye or felt upon the skin) beginning within about 5 minutes. Thus, the language “immediate skin tightening effect” or an “instantaneous skin tightening effect” means that when a layer the cosmetic composition having a wet thickness of about 2 mm is applied to a substrate under ambient conditions, an observable tightening or contraction is visually or sensorially noticeable within 5 minutes. In addition, a tightening or contraction can be quantified and defined as a measured internal stress of 8% or more.

The term “oil/oil-in-water emulsion” or “O/O/W” emulsion is interchangeable with the term “wax/oil-in-water emulsion” and “wax-particles/oil-in-water emulsion” and refers to a composition having three distinct phases: an aqueous carrier phase; and oil phase of droplets dispersed throughout the aqueous phase; and a wax phase of discrete wax particles dispersed throughout the aqueous carrier phase. The composition is like a typical oil-in-water emulsion with respect to the oil phase and the aqueous carrier phase but differs in that discrete wax particles are separately also dispersed throughout the aqueous carrier phase.

The term “emulsifier” (and “surfactant”) includes salts of the surfactant, to the extent they exist, even if not explicitly stated. In other words, whenever the disclosure refers to an emulsifier (or surfactants), it is intended that salts are also encompassed to the extent they exist, even though the specification may not specifically refer to a salt (or may not refer to a salt in every instance throughout the disclosure), for example, by using language such as “a salt thereof” or “salts thereof.” Sodium and potassium are common cations that form salts with emulsifiers/surfactants. However, additional cations such as ammonium ions, or alkanolammonium ions such as monoethanolammonium or triethanolammonium ions, may also form salts of emulsifiers and surfactants.

In the context of the instant disclosure, a “composition colorant” is a compound that colors the composition but does not have an appreciable coloring effect on hair. In other words, the composition colorant is included to provide a coloring to the composition for aesthetic appeal but is not intended to impart coloring properties to hair. Styling gels, for example, can be found in a variety of different colors (e.g., light blue, light pink, etc.) yet application of the styling gel to hair does not visibly change the color of the hair.

The expression “one or more” means “at least one” and thus includes individual components as well as mixtures/combinations.

The term “plurality” means “more than one” or “two or more.”

Other than in the operating examples, or where otherwise indicated, all numbers expressing quantities of ingredients and/or reaction conditions can be modified in all instances by the term “about,” meaning within +/−10% of the indicated number. For example, an amount of “about 1 wt.” can include an amount as low as 0.90 wt. % or as high as 1.1. Similarly, an amount of “about 50” can include an amount as low as 45 wt. % and as high as 55 wt. %.

Some of the various categories of ingredients identified for the cosmetic composition overlap. In such cases where overlap may exist and the cosmetic composition includes an overlapping ingredient (or more than one overlapping ingredient), an overlapping ingredient does not represent more than one claimed component. For example, titanium dioxide may be construed as both an “soft-focus powder” and a “pigment.” If a claimed cosmetic composition requires both a soft-focus powder and a pigment, the titanium dioxide will serve as only the soft-focus powder or as only as the pigment. Titanium dioxide does not simultaneously qualify as both the soft-focus powder and the pigment.

All percentages, parts, and ratios herein are based upon the total weight of the compositions of the present invention, unless otherwise indicated.

All ranges and values disclosed herein are inclusive and combinable. For examples, any value or point described herein that falls within a range described herein can serve as a minimum or maximum value to derive a sub-range, etc. Furthermore, all ranges provided are meant to include every specific range within, and combination of sub-ranges between, the given ranges. Thus, a range from 1-5, includes specifically 1, 2, 3, 4 and 5, as well as sub ranges such as 2-5, 3-5, 2-3, 2-4, 1-4, etc.

All components positively set forth in the instant disclosure can be negatively excluded from the claims and from the compositions and methods described throughout the disclosure In other words, the cleansing compositions of the instant disclosure may be free or essentially free of any one or more of the components positively set forth in the instant disclosure for possible inclusion into the cleansing compositions.

The term “substantially free” or “essentially free” as used herein means the specific material may be present in small amounts that do not materially affect the basic and novel characteristics of the claimed invention. For instance, there may be less than 1% by weight of a specific material added to a composition, based on the total weight of the compositions (provided that an amount of 1% or less by weight does not materially affect the basic and novel characteristics of the claimed invention). Similarly, when a composition is essentially free from a particular element, the composition may include 1 wt. % or less, 0.5 wt. % or less, 0.1 wt. % or less, 0.05 wt. % or less, 0.01 wt. % or less, or none of the specified material. For example, if a composition is “substantially free” or “essentially free” from ethanol, the composition may optionally include ethanol in an amount up to 1 wt. %, based on the total weight of the composition, provided an amount of 1 wt. % does not materially affect the basic and novel characteristics of the composition. Furthermore, the composition may optionally include 0.5 wt. % or less, 0.1 wt. % or less, 0.05 wt. % or less, 0.01 wt. % or less, or none of the ethanol.

All publications and patent applications cited throughout the disclosure are incorporated herein by reference in their entirety, and for any and all purposes, as if each individual publication or patent application were specifically and individually indicated to be incorporated by reference. In the event of an inconsistency between the present disclosure and any publications or patent application incorporated herein by reference, the present disclosure controls.

1. A cosmetic composition comprising:
(a) an aqueous carrier phase comprising:
(i) about 1 to about 8 wt. % of one or more hydrophilic film forming polymers;
(ii) about 1 to about 25 wt. % of one or more water soluble solvents;
(iii) about 40 to about 80 wt. % of water;
(b) an oil phase dispersed throughout the aqueous carrier phase, the oil phase comprising:
(i) about 1 to about 8 wt. % of one or more hydrophobic film forming polymers;
(ii) about 1 to about 15 wt. % of one or more volatile hydrocarbon oils;
(c) about 0.5 to about 5 wt. % of one or more emulsifiers; and
(d) about 1 to about 10 wt. % of discrete wax particles dispersed throughout the aqueous carrier phase;
wherein all weight percentages are based on a total weight of the composition.

2. The cosmetic composition of claim 1, wherein the composition provides an immediate skin tightening effect upon application to the skin.

3. The cosmetic composition of claim 1, wherein (b) (i) and (b) (ii) are in a weight ratio of about 1:1 to about 1:10 ((b) (i):(b) (ii)) and (a) (i) and (b) (i) are in a weight ratio of about 3:1 to about 1:3 ((a) (i):(b) (i)).

4. The cosmetic composition of claim 1, wherein the one or more hydrophilic film forming polymers are selected from polyvinylpyrrolidone (PVP) polymers, vinylpyrrolidone copolymers, carboxylic acid or carboxylate-based homo- and copolymers, saccharide-based polymers, non-saccharide-based polymers, and combinations thereof.

5. The cosmetic composition of claim 4, wherein at least one of the one or more hydrophilic film forming polymers is selected from polyvinylpyrrolidone, vinylpyrrolidone copolymers, or combinations thereof.

6. The cosmetic composition of claim 5, wherein the polyvinylpyrrolidone and the vinylpyrrolidone copolymers are selected from polyvinylpyrrolidone (PVP), polyvinylpyrrolidone (PVP)/vinyl acetate copolymer (PVP/VA copolymer), polyvinylpyrrolidone (PVP)/eicosene copolymer, PVP/hexadecene copolymer, VP/methacrylamide/vinyl imidazole copolymer, or combinations thereof.

7. The cosmetic composition of claim 1, wherein the one or more water soluble solvents are selected from glycerin, C₂-C₆ mono-alcohols, polyols, glycols, glycol-ethers, or combinations thereof.

8. The cosmetic composition of claim 1, wherein the one or more hydrophobic film forming polymers are selected from polyacrylic acids and polyacrylic acid esters, polymethacrylic acid and polymethacrylic acid esters, polyalkylene oxides, such as polyethylene oxide, polyvinylacetates, polyvinylalcohols, polyvinylacetatephthalates (PVAP), polyvinyl acetate copolymers, polycrotonic acids, pullulan, and pullulan compounds such as trialkylsiloxysilylcarbamoyl pullulan compounds, where the alkyl group comprises from 1 to 6 carbon atoms, or combinations thereof.

9. The cosmetic composition of claim 8, wherein at least one of the one or more hydrophobic film forming polymers is selected from trialkylsiloxysilylcarbamoyl pullulan with alkyl groups comprising 1 to 6 carbon atoms.

10. The cosmetic composition of claim 9, wherein the trialkylsiloxysilylcarbamoyl pullulan compound is trimethylsiloxysilylcarbamoyl pullulan.

11. The cosmetic composition of 1, wherein the one or more volatile hydrocarbon oils are selected from hydrogenated polyisobutene, isoparaffin, isohexadecane, isododecane, isodecane, isohexane, undecane, tridecane, dodecane, or combinations thereof.

12. The skin perfecting composition of claim 1, wherein the cosmetic composition includes one or more polymeric emulsifiers selected polyacrylamide and acryloyldimethyltaurate polymers.

13. The cosmetic composition of claim 1, wherein the discrete wax particles have a melting point above 50° C.

14. The cosmetic composition of claim 1, wherein the discrete wax particles have an average particle size of about 1 μm to about 20 1 μm.

15. The cosmetic composition of claim 1, further comprising one or more soft-focus powders.

16. The cosmetic composition of claim 1, wherein the one or more soft-focus powders are selected from talc, mica, titanated mica, alumina, aluminum silicate, silica, fumed silica, silica silylate, polyamide, poly(methyl (meth)acrylate), polyethylene, polymethylsilsesquioxane, nylon-12, cellulose, polylactic acid, or combinations thereof.

17. The cosmetic composition of claim 1, wherein the composition further comprises one or more pigments.

18. The cosmetic composition of claim 1, wherein the composition has a pH of about 5 to about 8.

19. A cosmetic composition comprising:
(a) about 65 to about 85 wt. % of an aqueous carrier phase comprising:
(i) about 1 to about 8 wt. % of one or more hydrophilic film forming polymers selected from polyvinylpyrrolidone (PVP), polyvinylpyrrolidone (PVP)/vinyl acetate copolymer (PVP/VA copolymer), polyvinylpyrrolidone (PVP)/eicosene copolymer, PVP/hexadecene copolymer, VP/methacrylamide/vinyl Imidazole copolymer, or combinations thereof; and
(ii) about 5 to about 10 wt. % of one or more water soluble solvents selected from glycerin, C₂-C₆ mono-alcohols, polyols, glycols, glycol ethers, or combinations thereof; and
(iii) about 40 to about 80 wt. % of water;
(b) about 3 to about 15 wt. % of an oil phase dispersed throughout the aqueous carrier phase, the oil phase comprising:
(i) about 1 to about 8 wt. % of one or more trialkylsiloxysilylcarbamoyl pullulan compounds;
(ii) about 2 to about 15 wt. % of one or more volatile hydrocarbon oils selected from polyisobutene, isoparaffin, isohexadecane, isododecane, isodecane, isohexane, undecane, tridecane, dodecane, or combinations thereof;
(c) about 0.5 to about 5 wt. % of one or more emulsifiers, wherein at least one of the one or more emulsifiers is a polymeric emulsifier selected from poloxamers (ethoxylated sorbitan), alkyl and polyalkyl ethers of poly(ethylene oxide), taurate copolymers, or combinations thereof;
(d) about 1 to about 10 wt. % of discrete wax particles dispersed throughout the aqueous carrier phase, wherein the discrete wax particles have an average particle size of about 1 μm to about 50 μm and a melting point greater than 55° C.; and
(e) optionally, about 0.1 to about 5 wt. % of one or more soft-focus powder;
(f) optionally, about 0.1 to about 5 wt. % of one or more pigments;
wherein the cosmetic composition provides an immediate skin tightening effect upon application to the skin, and
all weight percentages are based on a total weight of the composition.

20. A method for treating skin comprising applying the cosmetic composition of claim 1 to the skin.
